SEQUENCE LISTING (49 sequences)

SEQ ID NO: 1 - DNA, 22 bp, Artificial Sequence
  Primer
tgaatctgctttatttccacca

SEQ ID NO: 2 - DNA, 22 bp, Artificial Sequence
  Primer
cagccatagaaacaattcctca

SEQ ID NO: 3 - DNA, 20 bp, Artificial Sequence
  Primer
aaggggcacctctaccagtt

SEQ ID NO: 4 - DNA, 20 bp, Artificial Sequence
  Primer
ttcagtggccttgtgttctg

SEQ ID NO: 5 - DNA, 22 bp, Artificial Sequence
  Primer
ttcatattggtctttgccttga

SEQ ID NO: 6 - DNA, 20 bp, Artificial Sequence
  Primer
cgcccaaaatttgtgttctt

SEQ ID NO: 7 - DNA, 23 bp, Artificial Sequence
  Primer
gccaaaactcaaagaaagagtcc

SEQ ID NO: 8 - DNA, 20 bp, Artificial Sequence
  Primer
gtgtgcgcctgtttttacct

SEQ ID NO: 9 - DNA, 24 bp, Artificial Sequence
  Primer
aatagaccaccaagaactgacaaa

SEQ ID NO: 10 - DNA, 24 bp, Artificial Sequence
  Primer
tttccattttgcatgtactagacc

SEQ ID NO: 11 - DNA, 26 bp, Artificial Sequence
  Primer
caaaataaagatgacatggtagagga

SEQ ID NO: 12 - DNA, 20 bp, Artificial Sequence
  Primer
caaagtgcttgacgtcctga

SEQ ID NO: 13 - DNA, 25 bp, Artificial Sequence
  Primer
ctgtagattttcctatcaagggtga

SEQ ID NO: 14 - DNA, 20 bp, Artificial Sequence
  Primer
cgatcatccaattcaccaca

SEQ ID NO: 15 - DNA, 26 bp, Artificial Sequence
  Primer
tgaatatatgttaatgctctcgaacg

SEQ ID NO: 16 - DNA, 21 bp, Artificial Sequence
  Primer
agtccatttgcaagtttgtgg

SEQ ID NO: 17 - DNA, 22 bp, Artificial Sequence
  Primer
gtctgtctgtctagctcggtca

SEQ ID NO: 18 - DNA, 20 bp, Artificial Sequence
  Primer
ctgcatgcaatatgggtgat

SEQ ID NO: 19 - DNA, 27 bp, Artificial Sequence
  Primer
cataattattgcgatatttgtacagca

SEQ ID NO: 20 - DNA, 23 bp, Artificial Sequence
  Primer
aggaagaagaaaccaatccagtc

SEQ ID NO: 21 - DNA, 24 bp, Artificial Sequence
  Primer
ccaggagataagaagtgtgggata

SEQ ID NO: 22 - DNA, 21 bp, Artificial Sequence
  Primer
aagcagccatttcagatacca

SEQ ID NO: 23 - DNA, 27 bp, Artificial Sequence
  Primer
gaaggttctagtttatgctattgggta

SEQ ID NO: 24 - DNA, 19 bp, Artificial Sequence
  Primer
accaggggtgaagagagga

SEQ ID NO: 25 - DNA, 24 bp, Artificial Sequence
  Primer
cttgaataatcacgataggcaaga

SEQ ID NO: 26 - DNA, 20 bp, Artificial Sequence
  Primer
gcagaggaggcatcaagaag

SEQ ID NO: 27 - DNA, 26 bp, Artificial Sequence
  Primer
ttttgaatctaatggtttctctgttg

SEQ ID NO: 28 - DNA, 22 bp, Artificial Sequence
  Primer
caccaacatgcaacaaatcttc

SEQ ID NO: 29 - DNA, 24 bp, Artificial Sequence
  Primer
agaagaaaagaagctttcttgcac

SEQ ID NO: 30 - DNA, 20 bp, Artificial Sequence
  Primer
tcgagaggttccctgacaag

SEQ ID NO: 31 - DNA, 26 bp, Artificial Sequence
  Primer
aatgtaattccgttcataatttttcc

SEQ ID NO: 32 - DNA, 20 bp, Artificial Sequence
  Primer
caagaggcatgactcgaaca

SEQ ID NO: 33 - DNA, 403 bp, Carya illinoinensis
ggctgcttctggctcattgctttttgaatatcctctaatcatggtgttccacatgaaagtattgggtctactaatttcgtcaaaaaccttccgagcataagtcaagctgccaaaatctggggagatgctggaagccaggagcctgctcactgcgtttgggtctccaatgagacctgttttataaagtcgagcatgaatctgctttatttccaccatttttmaacatctttcaagcagacacagactctgtyctacatttgattctatggaggtctgattttgtagtttcactgaaatggtttccatggaatggttgatatgtgataaaattggtataggttcataacgagaagaagatgagaaaaatgaggaattgtttctatggctgtgattttattagaaa

SEQ ID NO: 34 - DNA, 104 bp, Carya illinoinensis
ccagttttgttaaggggcacctctaccagttagtttccatcttctgtctcttcatcttctgttctccaaactgaagaghragcagatytagaaaagtttaacca

SEQ ID NO: 35 - DNA, 500 bp, Carya illinoinensis
ctcaacccttctagctagctccaaaatattaaaaatggcaggcgctgatgattcagttccatttacacccagcaaactgtatccgaaaagcgatgaagagatcgccacgttcaaagaaatgaagaaagaacgaagtggtaaatgtttcttttatgtttttgctggaattgtcatccagtgcgttttcatattggtctttgccttgattacgctgcgtagtagaattcccgatgttaaggtaaggtcgattrcggtgaagaatctcaggtataataatggcacakcttcggcatcaatatctccttcttttaatgccactttggtcgccgaggtgaccattaagaacacaaattttgggcggtttaagttcgagaataccaccatgagtgtgctctatggaggcatgaacgtcgttggagatacaaaaataggacatgggagtgtcaaagctagagacacccagaagatgatcaatcttacagtggaggtgagatccaata

SEQ ID NO: 36 - DNA, 476 bp, Carya illinoinensis
gtcaaactgaactactcaggcggcagagagtaccgctcaaacaaaagagaaaaagaagaaagataacacgcacaaactaggcactcgagacaactcagaacccaagagattacacagctgatcagaaaaaagccaaaactcaaagaaagagtccgcggcctctaccgatccaagcagaagaagccaaaaacagaagtgatcttggatgcacctatcccagacagyaccttcactaatttggcaaatcaatctctacccaaggtaaaaacaggcgcacacacgcacctatatatatgtatatatatgtacacacaccacatgcagtaacggcaaagaacaacatgaacaagaagatgctgcataacccaagccaatatcgaaaaattcaagggaaccaaaaaacaggagcagctgcagccatgatgataagccgctaccacaaccccgtcctctcagcaaaccagtgcggttcattc

SEQ ID NO: 37 - DNA, 462 bp, Carya illinoinensis
gtcaaactgaactactcaggcggcagagagtaccgctcaaacaaaagagaaaaagaagaaagataacacgcacaaactaggcactcgagacaactcagaacccaagagattacacagctgatcagaaaaaagccaaaactcaaagaaagagtccgcggcctctaccgatccaagcagaagaagccaaaaacagaagtgatcttgagacagyaccttcactaatttggcaaatcaatctctacccaaggtaaaaacaggcgcacacacgcacctatatatatgtatatatatgtacacacaccacatgcagtaacggcaaagaacaacatgaacaagaagatgctgcataacccaagccaatatcgaaaaattcaagggaaccaaaaaacaggagcagctgcagccatgatgataagccgctaccacaaccccgtcctctcagcaaaccagtgcggttcattc

SEQ ID NO: 38 - DNA, 463 bp, Carya illinoinensis
ttccattcttttctttttggggtgggaaaataatctcaaaacttgacatggacatgctttggctttaggaggtttcactccctgtgacagactgagtaggtttgtagtgtgaccaattggtaaactaatagaccaccaagaactgacaaagctaacacagcatttagtctcatctagctgaatacagtctgagcacaaactcttggrccagracttcataataaaataaaaaccgaaatcctgatccctctatctgtctctcaagataataggtctagtacatgcaaaatggaaattaaatttacaagacatttccaccttataacgcacgaccctttcacttgtaagcttgcgactgatcttaataaaattatatcttcttctataggtactgctgaaacttgaaaattgatcacttgaactaccccccgagtatgcatttctttgtccaaggttcttcctc

SEQ ID NO: 39 - DNA, 500 bp, Carya illinoinensis
tctggcctatggtgggtaagggcagttgaggaatttctcaatgtcctacgctccagcccatatatgcatgtgcaacagaagatgtggagctctagccctcattcatcactggattccacaatcgatcagggtccatacgtaaatatcgcagatggaatggtggctgcaatgtgcaatgagcaaaataaagatgacatggtagaggaattacctgaaccgccgaagaattctgatgaagaagataataaaarggcttttgaagcaaaacaagatgagcaccagacagaagcaaaagatgatgtttccccggatattgcagaaatcaaaaaccaaactacctttgttcaggacgtcaagcactttgatgcaatttctccagttcgaagatgcttagatctgctgtctcggcagaataggtgttccatatggttgcttgcttacattgctattatatcaagttggccattggtaggctcagctctgcaaaccgtcttcagaaa

SEQ ID NO: 40 - DNA, 500 bp, Carya illinoinensis
agcagcctgcttacaggataggtgtggagtcaaaaatttctaaaactaatgatagacaggagaacatttccttagacaagtctgcattaaggtctgtagattttcctatcaagggtgaaggtcgagttgttaagaatcttgttagaaatgaggagaaattaaaatcttctcatgattgtaatgaattggaggataggccatctgagaatgtaatttgtgtcagaattgaggagaaagtaaaatctattcaygactgtggtgaattggatgatcgaccgtctaagaaggcaaagttggattgttctgttaaagtatttggtgataagaaagagaagagtgtgcggaagttacctgttgatttgagtcgtcatgatgcaaggggtgtaggaactgtaattgattttgaggataaatctaggcttaaacttgttaaggattctcgaggggttgaaaatggccctttgaagaagttgaagcttgatgagaagacaaagcctttt

SEQ ID NO: 41 - DNA, 500 bp, Carya illinoinensis
agagaccccggacaaccattttgataaaatttgctgaagaggtactacgaagagaagcaaggttaggttaatgtgtgtgcctggatttattacatgtaatgtttggatcgatgcttagcaagaacgtgatgaatatatgttaatgctctcgaacgacttgatggactctggaacatttttggccattcacttgccatttctgtcactctggccaatttgtgtctatcatagatttgtattgatatatactmgacttcatgccacaaacttgcaaatggacttgaatgtaatttatggagggggctctcttttattttcatcacacagccgtaggggaaaataattttcttgttctcagaaaatgtattctttactgttctggttgatttaggtcataactagaggtgttatttacatggtaagtaggattttagtttcagccgaaatcaaaagtactgatctcatagcttccatgaacttggacgaacgatctactcagt

SEQ ID NO: 42 - DNA, 286 bp, Carya illinoinensis
aagtagtctttatatctgtctgtctgtctgtctagctcggtcatgtttaaatcgtyatkgatgtttgatactttttcagacattgatattacctttctataatgtcttctacggtgctgttaatatacgaaaattaagtcctatagatgatgattttatcacccatattgcatgcagcaaacatgtaaatacacgaggctctgaatgtcctagtattgttgccgtttctgcgtatatcttatttatttgatcatcattctcatcatcatcatatatatatatatat

SEQ ID NO: 43 - DNA, 500 bp, Carya illinoinensis
tcaccaaatgcttttccatgggaaatgaggccgctcatgtggatacaatgcatggtaaaacagtctcttcaaatagtactctttctcagggaaaattacaatgactttatcacactcacattacgtagcataattattgcgatatttgtacagcaggtacaggttgcagatcggctgcaatttgttgcaagtccccttaatttcattttctttcmgmttaaactacaaatagttcaatgtagtggawtgahgttgcacgtgtaaactcgaaatatgactggattggtttcttcttcctttctatatttgccaacttgctgagttcagattgtgcatttgttggaatattttgtacccttcctatgtgcccaaatggttttctttttctttttctattttttctcgtttctttcaatgaaatcttatcaaagaaggaaaataatctcatttcatctgaaatttagctttaataacttcatgtgtacctaattacttttttg

SEQ ID NO: 44 - DNA, 500 bp, Carya illinoinensis
cccaacattgtgattgacatcaaaagtgaaaaatttcagtgtttggaagggatattcttgatgatgtttagatggttctgctcttaagatgttaaagaatcctaattggctttagaaggaaaaaccatgccaggagataagaagtgtgggatatatttctgtgatagtttgattggagtatctaacacgcggcacatatataaagacaaagaaactcctgatattcttatctttgccactatygttaaagrgttcaagtggtggatcakgttgttgcagtggtatctgaaatggctgcttctttagcaagagttgttaactcatcacaagcttctcagctgcacccagtcatatcttttactggacaccagagcagttgccagtcatctctgtttagtgattctagaggtcaacttatagggtggactggacatgtgttctggttcctttaaatatcctcacaatttctctcaaactgttcctcttattttagtaagatc

SEQ ID NO: 45 - DNA, 360 bp, Carya illinoinensis
tacctcaaaagatgagaaggctgtttgactactgctttcatatggaatctcagactatatctggcgaggcactatattattgactacgaatgattgtactctgaaggatgatacatatctctctcccatttgttactttatttttgttacgtcatttgttacttattatagaaggttctagtttatgctattgggtatatacctccatatttcttctaccatgactatatcttaataacaagttcaatagraattctaatagcaataaaggcttctttcttgtgtgtccaaccatgatttcctctcttcacccctggttggatcatgcatgcaaaggagtagagtttataaaaaaaaaaa

SEQ ID NO: 46 - DNA, 500 bp, Carya illinoinensis
ctgctcacactctacattgagaagtggggggaaaaagagtggaacaatctacttttttgttacctgcatggatacttttttatgatcctaaagcctacaaaactttgaaaataaggaaagattactgtaaaagtcttgaataatcacgataggcaagatcattaacttatacttgaaaggcttaatgtgtctgctcatgaaacatcaaaccgaacaacgatacaagtgatattaycagaacttcctcgggyataagcttctgtaacaagtcttcttgatgcctcctctgcatttgatatatcctgtactaaagccacagcatccttattcgatatgacattccaaagtccatcactagcaagaattataaaatctacaccatcaatttcttcttcctggatttctggctcagcaacaacatattgctttagtagtttgtctccaaatgcacgagaaatggcaagaacaccaccaaccctccaagtgccagcccaaatgat

SEQ ID NO: 47 - DNA, 466 bp, Carya illinoinensis
tttgattattgcaaatttctcgaggtaagagcaacgattttgaaatatctgggcctgccctttggctgcccagaaaaatgaacgaaattgaagaatgttttgaatctaatggtttctctgttgttttcgtctttgaaaaataatgtctagataatctcgactgagatttgggattctgttttctcttatttcctcggcaaacaaacagagagtcmattttcttttttctaaagaamatttgttgcatgtmggtgtatttagcagatcaatgtgaagttttatcttcggaatgtgcttgcgtttggtatttgatgtggaaatccatttattcttagaattttcctctctcttttttttttttttccctctatcttgctcatttggatatgttgcttaattattgaaatttaatctgacgtgattgattggtgtctaattggatatggttgctgagaaaacggaaggg

SEQ ID NO: 48 - DNA, 500 bp, Carya illinoinensis
atatttaaagaagactcaggaaaataaatattcttaagtgataacaaagtcaatttcattcataattagctctgaccctcagagctttggaaaggaggaggaaatccgactagccattgacattaggttgtgacagatcgagaagaaaagaagctttcttgcactttccgtgcattcaaatgatgggaaaagtcatgagtccaccactaactactttcctacaggagatgtttcrccccaactaccattcwgccttttaacaagattacaccatgagatttcaaagttacttgtcagggaacctctcgaaggcagggcccttcaaactcacccttatagagtaaagcccggtcccatacaccgtaccctcaaaagtttccttatatgaaactggttaaatcgttggctttgcgcatagtcattatttgccatggaatattgtagattgggaggaagctgtggatggagtaaatcacataaaatctgagctgagcttgagt

SEQ ID NO: 49 - DNA, 500 bp, Carya illinoinensis
gtcctgtaaaagtaggatttcagggaatttggaatcggatttcttattagttttttcgttatttgtttcttagttttgggtgtatatatccctctctctgtacatggaacttcaactcttttcttatttgctcatctcagtttatgataagtgacaattcaattaattaatgtaattccgttcataatttttcccatatctcgatttgttgaactctcaaaagctcctcacctgtcttgcacacagaattrccatgcccacactatcatcatgagttggcgtaatagcctcgtgagcggcgcggcagggactgatgccaaagtctgaayttgttcgagtcatgcctcttgcgcacgtaaagattgttcccgagtggggtgggtatgtgacggttttaacctgtttagtacaatacccagtgtccacctccacttccatgatcagatgcacgagcaagtgccgatcgactaatagttgcccgatattatgatattattttt

(19) United States
(12) Patent Application Publication
(10) Pub. No.: US 2017/0268072 A1
(43) Pub. Date: Sep. 21, 2017

(54) METHODS FOR IDENTIFYING PECAN TREE CULTIVARS

(71) Applicant: The Samuel Roberts Noble Foundation, Inc., Ardmore, OK (US)

(72) Inventors: Maria J. Monteros, Ardmore, OK (US); Yanina Alarcon, Ardmore, OK (US); Charles Rohla, Ardmore, OK (US)

(21) Appl. No.: 15/465,434

(22) Filed: Mar. 21, 2017

Related U.S. Application Data
(60) Provisional application No. 62/311,201, filed on Mar. 21, 2016.

(51) Int. Cl. C12Q 1/68

(52) U.S. Cl. CPC C12Q 1/6895; C12Q 2600/156

(57) ABSTRACT

The present invention provides compositions and methods for identifying cultivars of pecan (Carya illinoinensis K.).

### REFERENCE TO RELATED APPLICATIONS

This application claims the benefit of U.S. Provisional Patent Application Ser. No. 62/311,201, filed Mar. 21, 2016, which is herein incorporated by reference in its entirety.

### INCORPORATION OF SEQUENCE LISTING

The sequence listing that is contained in the file named “NBLE092US_ST25.txt,” which is 21 kilobytes as measured in Microsoft Windows operating system and was created on Mar. 21, 2017, is filed electronically herewith and incorporated herein by reference.

### FIELD OF THE INVENTION

The present invention relates generally to the field of molecular biology. More specifically, the invention relates to methods of identifying pecan tree cultivars.

### BACKGROUND OF THE INVENTION

Pecan (Carya illinoinensis K.) is a valuable specialty crop. The pecan industry propagates pecan cultivars clonally through the use of graftwood. As clones, different trees from the same cultivar should be genetically identical. Pecan cultivars are currently identified visually, which can lead to different outcomes based on the individual performing the identification, challenges to identify subtle differences, and problems with variation in nut characteristics due to environmental factors such as water and nutrients, or simply based on historical assessment, wherein all trees that are stated to be derived from a particular cultivar are assumed to be genetically identical and derived from the stated cultivar. However, there is mounting evidence that individual trees allegedly from the same cultivar have important differences between them, for example susceptibility to the pecan scab disease, suggesting that these trees are not genetically identical to each other. Therefore, these trees could also differ in their agronomic performance, nut production (yield), disease resistance and nut characteristics. Unfortunately, pecan is currently lacking effective and reliable genome-wide level genomic resources to identify specific cultivars. Accordingly, methods and compositions for genetic identification of pecan cultivars would represent a tremendous advance in the field.

### SUMMARY OF THE INVENTION

The present invention provides methods and compositions for identifying a cultivar of a pecan tree. In one embodiment, the method comprises detecting a polymorphism, for example a first insertion/deletion or single nucleotide polymorphism selected from the group of insertion/deletion and single nucleotide polymorphisms listed in Table 3, from a sample of nucleic acids from the pecan tree, wherein the presence of the first insertion/deletion or single nucleotide polymorphism identifies the cultivar of the pecan tree. In certain embodiments, the cultivar is Alley, Amling, Apache, Barton, Brooks, Burkett, Byrd, Caddo, Candy, Cape Fear, Carmichael, Carter, Cheyenne, Choctaw, Clark, Creek, Curtis, Dependable 6-2, Dependable 8-3, Desirable 5-2, Eclipse, Elliott, Evers, Excel, Forkert, Gafford, Giles, Halbert, Humble, Jackson BW, Jackson LA, Jenkins, Kanza, Kiowa, Lakota, Mahan, Major, Mohawk, Moreland, Nacono, Oconee, Odom, Pawnee, Podsednik, Riverside, Russell, Schley, Shawnee, Starking Hardy Giant (SHG), Shoshoni, Sioux, Stuart, Success, Sumner, Syrup Mill, VC168, Western. Schley (W. Schley), Wichita, Witte, or Zinner.

In particular embodiments the first insertion/deletion or single nucleotide polymorphism is detected by amplification of a first nucleic acid segment comprising the first single nucleotide polymorphism from the sample of nucleic acids, and analysis of said first nucleic acid segment by high-resolution melting or sequencing to detect the first insertion/deletion or single nucleotide polymorphism. In certain embodiments, the first nucleic acid segment is sequenced directly to detect the first insertion/deletion or single nucleotide polymorphism. In some embodiments the first insertion/deletion or single nucleotide polymorphism is within a locus in the genome of the pecan tree having a nucleotide sequence of any one of SEQ ID NOs:33-49. In other embodiments the first insertion/deletion or single nucleotide polymorphism is within a locus in the genome of the pecan tree flanked by a pair of oligonucleotide primers of sufficient length to hybridize specifically to any one of SEQ ID NOs:33-49. In particular embodiments the pair of oligonucleotide primers is selected from the group consisting of SEQ ID NO:1 and SEQ ID NO:2, SEQ ID NO:3 and SEQ ID NO:4, SEQ ID NO:5 and SEQ ID NO:6, SEQ ID NO:7 and SEQ ID NO:8, SEQ ID NO:9 and SEQ ID NO:10, SEQ ID NO:11 and SEQ ID NO:12, SEQ ID NO:13 and SEQ ID NO:14, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:23 and SEQ ID NO:24, SEQ ID NO:25 and SEQ ID NO:26, SEQ ID NO:27 and SEQ ID NO:28, SEQ ID NO:29 and SEQ ID NO:30, SEQ ID NO:31 and SEQ ID NO:32.

In additional embodiments, the method further comprises detection of a second single nucleotide polymorphism by amplification of a second nucleic acid segment comprising the second single nucleotide polymorphism from the sample of nucleic acids, and analysis of the second nucleic acid segment by high-resolution melting or sequencing to detect the second single nucleotide polymorphism. In certain embodiments, the second nucleic acid segment is sequenced directly to detect the second single nucleotide polymorphism. In other embodiments, the method further comprises detection of a third single nucleotide polymorphism by amplification of a third nucleic acid segment comprising the third single nucleotide polymorphism from the sample of nucleic acids, and analysis of the third nucleic acid segment by high-resolution melting or sequencing to detect the third single nucleotide polymorphism. In certain embodiments, the third nucleic acid segment is sequenced directly to detect the third single nucleotide polymorphism. In yet other embodiments, the method further comprises detection of a fourth single nucleotide polymorphism by amplification of a fourth nucleic acid segment comprising the fourth single nucleotide polymorphism from the sample of nucleic acids, and analysis of the fourth nucleic acid segment by high-resolution melting or sequencing to detect the fourth single nucleotide polymorphism. In certain embodiments, the fourth nucleic acid segment is sequenced directly to detect the fourth single nucleotide polymorphism. In still other embodiments, the method further comprises detection of a fifth single nucleotide polymorphism by amplification of a fifth nucleic acid segment comprising the fifth single nucleotide polymorphism from the sample of nucleic acids, and analysis of the fifth nucleic acid segment by high-resolution melting or sequencing to detect the fifth single nucleotide polymorphism. In certain embodiments, the fifth nucleic acid segment is sequenced directly to detect the fifth single nucleotide polymorphism.

The present invention also provides kits for determining a cultivar of a pecan tree. In some embodiments the kit comprises at least a first pair of oligonucleotide primers of sufficient length to hybridize specifically to any one of SEQ ID NOs:33-49. In particular embodiments the at least a first pair of oligonucleotide primers is selected from the group consisting of SEQ ID NO:1 and SEQ ID NO:2, SEQ ID NO:3 and SEQ ID NO:4, SEQ ID NO:5 and SEQ ID NO:6, SEQ ID NO:7 and SEQ ID NO:8, SEQ ID NO:9 and SEQ ID NO:10, SEQ ID NO:11 and SEQ ID NO:12, SEQ ID NO:13 and SEQ ID NO:14, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:23 and SEQ ID NO:24, SEQ ID NO:25 and SEQ ID NO:26, SEQ ID NO:27 and SEQ ID NO:28, SEQ ID NO:29 and SEQ ID NO:30, SEQ ID NO:31 and SEQ ID NO:32. In certain embodiments the kit comprises all of the following oligonucleotide primer sets: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; SEQ ID NO:15 and SEQ ID NO:16; SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; SEQ ID NO:31 and SEQ ID NO:32, wherein the cultivar is Alley, Amling, Apache, Barton, Brooks, Burkett, Byrd, Caddo, Candy, Cape Fear, Carmichael, Carter, Cheyenne, Choctaw, Clark, Creek, Curtis, Dependable 6-2, Dependable 8-3, Desirable 5-2, Eclipse, Elliott, Evers, Excel, Forkert, Gafford, Giles, Halbert, Humble, Jackson BW, Jackson LA, Jenkins, Kanza, Kiowa, Lakota, Mahan, Major, Mohawk, Moreland, Nacono, Oconee, Odom, Pawnee, Podsednik, Riverside, Russell, Schley, Shawnee, Starking Hardy Giant (SHG), Shoshoni, Sioux, Stuart, Success, Sumner, Syrup Mill, VC168, Western Schley (W. Schley), Wichita, Witte, or Zinner. In some embodiments the kit further comprises instructions for determining the cultivar of the pecan tree. In certain aspects, the kits can be used to determine that an unknown pecan tree is not any of the above-listed cultivars.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein.

FIG. 1: Gene ontology assignment for pecan transcriptome sequences from two pecan cultivars based on the black walnut (Juglans nigra L.) genome annotation.

FIG. 2: Categories of candidate SNPs identified through alignment of sequences from the Pawnee vs. Kanza pecan cultivars. Category 1: Kanza homozygous (AA) vs. Pawnee homozygous (CC), 11,910 SNPs. Category 2: Kanza heterozygous (AC) vs. Pawnee homozygous (AA), 14,277 SNPs. Category 3: Kanza homozygous (AA) vs. Pawnee heterozygous (AC), 8,832 SNPs. Category 4: Kanza heterozygous (AC) vs. Pawnee heterozygous (AC), 8,398 SNPs. Total: 43,417 SNPs.

FIG. 3: High resolution melting peaks from candidate SNPs between Pawnee (red) and Kanza (grey). FIG. 3A: Category 1 SNP CI0001 confirms the presence of a single SNP between Pawnee (GG) and Kanza (AA). FIG. 3B: Category 2 SNP CI0030 confirms Pawnee (TT), the predicted Kanza (CC) appears to have more than one SNP in the target amplicon. FIG. 3C: Category 3 SNP CI0091 confirms the presence of a single SNP Pawnee (CG) and Kanza (CC). FIG. 3D: The overlapping melting profiles for CI0094 of the Pawnee and Kanza trees indicate the absence of a predicted SNP.

FIG. 4: High resolution melting peaks from validated SNPs on the ORTET pecan samples. The ORTET represents the original plant or as close to the “Mother plant” from which the members of a clone have descended. SNPs shown here: CI0001 (Panel 1), CI0008 (Panel 2), CI0012 (Panel 3), CI0015 (Panel 4), CI0018 (Panel 5), and CI0025 (Panel 6).

FIG. 5: High resolution melting peaks from validated SNPs on the ORTET pecan samples. SNPs shown here: CI0029 (Panel 1), CI0043 (Panel 2), CI0048 (Panel 3), CI0051 (Panel 4), CI0053 (Panel 5) and CI0058 (Panel 6).

FIG. 6: High resolution melting peaks from validated SNPs on the ORTET pecan samples. SNPs shown here: CI0062 (Panel 1), CI0081 (Panel 2), CI0084 (Panel 3), and CI0089 (Panel 4).

### BRIEF DESCRIPTION OF THE SEQUENCE LISTING

SEQ ID NOs:1-32—DNA sequences of forward and reverse primers for identifying cultivars of pecan.

SEQ ID NOs:33-49—DNA sequences of regions surrounding insertion/deletion or single nucleotide polymorphisms identified in pecan cultivars.

### DETAILED DESCRIPTION OF THE INVENTION

The present invention provides compositions and methods of determining a cultivar of a pecan tree, comprising detecting one or more single nucleotide polymorphism(s) or insertion/deletions from a sample of nucleic acids from the pecan tree, wherein the presence of the one or more single nucleotide polymorphism(s) identifies the cultivar of the pecan tree.

Pecan (Carya illinoinensis (Wangenh.) K. Koch), perhaps one of the most important horticultural crops, is a member of the Juglandaceae family. The family Juglandaceae contains both walnuts (Juglans spp.) and hickories (Carya spp.). The genus Carya has 20 species including pecan, 13 of which are native to the United States (Grauke and Thompson, 1996). Pecans are indigenous to a large area in North America stretching from southern Illinois down the Mississippi river valley and south into the northern area of Mexico, and west to the branches of the Llano and San Saba rivers. Pecan is a long-lived heterodichogamous tree species with male and female flowers on the same tree maturing at separate times (Thompson, 1985) that promotes cross-pollination with wind-dispersed pollen (Wood, et al., 1997). Therefore, pecan exhibits high heterozygosity and severe inbreeding depression. Commercial pecan orchards were established early on by selecting open-pollinated nuts from a mature tree that was selected based on specific desirable traits. However, each resulting nut from the same tree was genetically different because it was open pollinated with pollen from any of the male flowers in the orchard. Vegetative propagation methods were later developed, and the first record of tree nurseries selling grafted pecan trees was documented in 1879 (Crane, et al., 1937). Modified techniques for grafting were later developed (McHatton, 1957; Wood, 1990) and used in the commercial pecan industry to generate and sell named improved cultivars that were clonally propagated.

The pecan industry propagates pecan cultivars clonally through the use of graftwood. However, the inventors have discovered by genetic evaluation of multiple individual trees from the same cultivar the presence of genetic differences between them, suggesting that these trees are not genetically identical to each other. Therefore, these trees could also differ in their agronomic performance, disease resistance, nut yields and other nut characteristics including nutrient composition.

With the present invention, for the first time, it is possible to define pecan trees at the genetic level, and correctly identify the cultivar of any particular pecan tree. Particular polymorphisms or groups of polymorphisms at the DNA level were found to be determinative for assigning a cultivar designation. In certain embodiments, the present invention provides information to growers on the cultivar or cultivars currently grown in an existing orchard, enables growers to make educated decisions about how to manage their orchard (for example, to plan for fungicide applications if the trees are susceptible to pecan scab), plan for the future, and clearly articulate the product of their trees, given that the nuts from different cultivars may have different nutrient profiles. In additional embodiments, the present invention allows growers to identify pecan cultivars growing in an orchard that they are considering purchasing. In further embodiments, the present invention allows for the generation of a DNA profile of key pecan cultivars that have the most desirable characteristics (including, but not limited to, nut quality and pecan scab resistance) so that a grower can ensure the genetics of the trees under consideration for purchase, or to assist in breeding efforts. In other embodiments, the present invention allows interested nurseries to sell DNA-certified′ cultivars to validate the genetic identity of the trees being sold to growers, which translates to conservation of genetic identity, premium pricing for highly sought after cultivars, higher consumer confidence and a premium per tree for certification of the genetics.

DNA primers are isolated nucleic acids that are annealed to a complementary target DNA strand by nucleic acid hybridization to form a hybrid between the primer and the target DNA strand, then extended along the target DNA strand by a polymerase, e.g., a DNA polymerase. A DNA primer pair or a DNA primer set of the present disclosure refer to two DNA primers useful for amplification of a target nucleic acid sequence, e.g., by the polymerase chain reaction (PCR) or other conventional nucleic acid amplification methods.

DNA primers are generally 15 nucleotides or more in length, often 18 nucleotides or more, 24 nucleotides or more, or 30 nucleotides or more. In certain embodiments the DNA primers are 16 nucleotides in length, 17 nucleotides in length, 19 nucleotides in length, 20 nucleotides in length, 21 nucleotides in length, 22 nucleotides in length, 23 nucleotides in length, 25 nucleotides in length, 26 nucleotides in length, 27 nucleotides in length, 28 nucleotides in length, or 29 nucleotides in length. Such primers are selected to be of sufficient length to hybridize specifically to a target sequence under high stringency hybridization conditions. Preferably, primers according to the present disclosure have complete sequence similarity with the target sequence, although probes differing from the target sequence that retain the ability to hybridize to target sequences may be designed by conventional methods.

The nucleic acid primers of the present disclosure hybridize under stringent conditions to a target DNA molecule. Any conventional nucleic acid hybridization or amplification method can be used to identify the presence of an insertion/deletion or single nucleotide polymorphism in a sample. Nucleic acid molecules, also referred to as nucleic acid segments, or fragments thereof are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook, et al., Molecular Cloning: A Laboratory Manual, 2nd Edition, Cold Spring Harbor Laboratory, Cold Spring Harbor (1989), and by Haymes, et al., In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. In order for a nucleic acid molecule to serve as a primer it need only be sufficiently complementary in sequence to be able to form a stable double-stranded structure under the particular solvent and salt concentrations employed.

Appropriate stringency conditions that promote DNA hybridization, for example, include 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. In one embodiment, a DNA primer of the present disclosure will specifically hybridize to one or more of the nucleic acid molecules set forth in SEQ ID NOs: 33-49, or complements thereof or fragments of either under moderately stringent conditions, for example at about 2.0×SSC and about 65° C. In another embodiment, a DNA primer of the present disclosure will specifically hybridize to one or more of the nucleic acid molecules set forth in SEQ ID NOs:33-49 or complements or fragments of either under high stringency conditions. In certain aspects of the present disclosure, a DNA primer of the present disclosure has the nucleic acid sequence set forth in SEQ ID NOs:1-32 or complements thereof or fragments of either.

Regarding the amplification of a target nucleic acid sequence (e.g., by PCR) using a particular amplification primer pair, “stringent conditions” are conditions that permit the primer pair to hybridize only to the target nucleic acid sequence, for example any one of SEQ ID NOs:33-49, to which a primer having the corresponding wild-type sequence (or its complement) would bind and preferably to produce a unique amplification product, the amplicon, in a DNA thermal amplification reaction. The term “specific for (a target sequence)” indicates that a primer hybridizes under stringent hybridization conditions only to the target sequence in a sample comprising the target sequence.

As used herein, “amplified DNA” or “amplicon” refers to the product of nucleic acid amplification method directed to a target nucleic acid molecule that is part of a nucleic acid template. The amplicon may range in length from the combined length of the primer pair plus one nucleotide base pair, preferably plus about fifty nucleotide base pairs, more preferably plus about two hundred-fifty nucleotide base pairs, and even more preferably plus about four hundred-fifty nucleotide base pairs or more. Alternatively, a primer pair can be derived from genomic sequence on both sides of the insertion/deletion or single nucleotide polymorphism so as to produce an amplicon that includes the entire insertion/deletion or single nucleotide polymorphism. A member of a primer pair derived from the pecan genomic sequence adjacent to the insertion/deletion or single nucleotide polymorphism is located a distance from the insertion/deletion or single nucleotide polymorphism, this distance can range from one nucleotide base pair up to about one thousand nucleotide base pairs or more. The use of the term “amplicon” specifically excludes primer dimers that may be formed in the DNA thermal amplification reaction.

Nucleic acid amplification can be accomplished by any of the various nucleic acid amplification methods known in the art, including the polymerase chain reaction (PCR). Amplification methods are known in the art and are described, inter alia, in U.S. Pat. Nos. 4,683,195 and 4,683,202 and in PCR Protocols: A Guide to Methods and Applications, ed. Innis, et al., Academic Press, San Diego, 1990. These methods as well as other methods known in the art of DNA amplification may be used in the practice of the present disclosure.

DNA kits that are based on DNA amplification methods contain DNA primer molecules that hybridize specifically to a target DNA and amplify a diagnostic amplicon comprising one or more insertion/deletion or single nucleotide polymorphism under the appropriate reaction conditions. A kit that contains DNA primers that are homologous or complementary to any portion of the target nucleic acid sequences as set forth in any of SEQ ID NOs:33-49 or to any portion thereof. DNA molecules useful as DNA primers can be selected from the disclosed target nucleic acid sequences as set forth in any of SEQ ID NOs:33-49 by those skilled in the art of DNA amplification.

The following example is included to demonstrate preferred embodiments of the invention. It should be appreciated by those of skill in the art that the techniques disclosed in the example that follows represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Genome-Wide SNP Discovery in Pecan (Carya illinoinensis K.) and Characterization of Pecan Germplasm

This example describes the generation of pecan transcriptome, the mining of those sequences for genome-wide SNP discovery in pecan, and utilization of a subset of validated SNPs to genotype pecan cultivars from the Carya species germplasm collection for characterization of named cultivars at the DNA level.

The pecan cultivars Pawnee and Kanza differ for various agronomic characteristics in the field including nut size and disease response to pecan scab. The transcriptome sequences covered a range of functional categories based on the black walnut (Juglans nigra L.) annotation in the absence of a sequenced and annotated pecan genome. A total of 43,417 candidate SNPs were identified between the two pecan cultivars. A subset of candidate SNPs were evaluated and validated using high resolution melting (HRM) analysis in the original pecan cultivars used for SNP discovery. Sixteen of the validated SNPs were used to survey a set of sixty two pecan cultivars and those generated specific marker profiles capable of distinguishing between sixty of the pecan cultivars evaluated, at the genetic level.

Methods

Plant Materials

One year old trees from the pecan cultivars ‘Pawnee’ and ‘Kanza’ (source: WoMack Nursery, DeLeon, Tex.) were grafted onto three year old ‘Apache’ rootstock and planted at the Samuel Roberts Noble Foundation's McMillan research farm in Marshall County, Okla. All trees were bare root trees that were root pruned before planting. Vegetation was controlled with herbicides to maintain a 1.8-meter wide vegetation-free strip centered on the tree rows. Trees were planted on a 10.6 meter spacing. All trees were irrigated with a solid set sprinkler system. Trees were planted on a Bastrop fine sandy loam soil (fine-loamy, mixed, active, thermic, udic Paleustalfs) soil. Plant tissues were sampled for three Pawnee trees (tree coding=NF07PEC0001-0038, NF07PEC0001-0039, NF07PEC0001-0040 with the corresponding sample coding=14P38, 14P39 and 14P40), and three Kanza trees (NF07PEC0001-0018, NF07PEC0001-0019, NF07PEC0001-0020 with the sample coding 14K18, 14K19 and 14K20, respectively). Terminal tips and approximately 30 leaflets ranging from young to more mature tissue were harvested from multiple tree positions. Samples were harvested in the field, placed on dry ice for transport back to the lab, and stored in a −80° C. freezer until processing.

DNA Extraction Protocol of the Pecan Cultivars Corresponding to the ORTET Samples

Carya germplasm leaf samples were collected from sixty two ORTET trees representing pecan cultivars at the USDA-ARS Texas A & M facility in College Station, Tex. Samples were collected on ice, lyophilized for four days and DNA extracted using the Plant DNAeasy Mini Kit (Qiagen, Valencia, Calif.) using the following standard procedure (based on the manufacturer's instructions): 1) Preheat a water bath or heating block to 65° C.; 2) Use TissueLyser for tissue disruption as follows: place 15 mg of the lyophilized sample tissue into a 2 ml safe-lock microcentrifuge tube, together with a 3 mm tungsten carbide bead; 3) Place the tubes into the TissueLyser Adapter Set 2×24, and fix into the clamps of the TissueLyser. Immediately grind the samples for 1 min at 30 Hz, and do this three times; 4) Add 400 μl Buffer AP1 and 4 μl RNase A stock solution (100 mg/ml) to a maximum of 15 mg (dried) disrupted plant tissue and vortex vigorously. Vortex at maximum speed for 1 min or more to remove any clumps; 5) Incubate the mixture for 10 min at 65° C. to lyse the cells. Mix two or three times during incubation by inverting the tube; 6) Add 130 μl Buffer AP2 to the lysate, mix, and incubate for 5 min on ice to precipitate proteins and polysaccharides; 7) Centrifuge the lysate for 7 min at 20,000×g (14,000 rpm); 8) Pipet the lysate into the QIAshredder Mini spin column placed in a 2 ml collection tube and centrifuge for 2 min at 20,000×g (14,000 rpm); 9) Transfer the flow-through fraction into a new tube without disturbing the cell-debris pellet. Typically 300 μl of lysate is recovered; 10) Add 1.5 volumes of Buffer AP3/E to the cleared lysate, and mix immediately using a pipette; 11) Transfer 650 μl of the previous mixture, including any precipitate that may have formed, into the DNeasy Mini spin column placed in a 2 ml collection tube. Centrifuge for 1 min at 13,000×g, and discard the flow-through; 12) Transfer the remaining mixture and centrifuge for 2 min at 13,000×g, and discard the flow-through. Discard flow-through and collection tube; 13) Place the DNeasy Mini spin column into a new 2 ml collection tube, add 500 μl Buffer AW, and centrifuge for 1 min at 13,000×g. Discard the flow-through; 14) Add 500 μl Buffer AW to the DNeasy Mini spin column, and centrifuge for 2 min at 20,000×g to dry the membrane. Discard flow-through; 15) Centrifuge 1 min at 13,000×g and discard any remaining flow-through; 16) Transfer the DNeasy Mini spin column to a 1.5 ml tube, and pipet 50 μl Buffer AE directly onto the DNeasy membrane. Incubate for 15 min at room temperature (15-25° C.), and then centrifuge for 1 min at 13,000×g to elute. DNA was quantified using a NanoDrop Spectrophotometer ND-100 (NanoDrop Technologies, Willington, Del.) and DNA dilutions were made to a working concentration of 5 ng/μl.

cDNA Preparation and Sequencing

Total RNA was extracted from 50-75 mg of tissue using Sigma Spectrum Plant Total RNA kit with a 40° C. incubation, using 750 μl of binding buffer and Protocol A and quantified using a NanoDrop Spectrophotometer ND-100 (NanoDrop Technologies, Willington, Del.). Total RNA integrity was assessed using a Bioanalyzer 2100 (Agilent, Santa Clara, Calif.). Messenger RNA (mRNA) isolation was performed using the oligo (dT) magnetic beads. The mRNA was fragmented into short fragments (200-500 bp) with a fragment buffer treatment. The first strand cDNA was synthesized by random hexamer-primer with the mRNA fragment as templates. Buffers, dNTPs, RNase H and DNA polymerase I were used to synthesize the second strand. The double strand cDNAs were purified with the Qiaquick PCR extraction kit and used for end-repair and base ‘A’ addition. Finally, sequencing adaptors were ligated to the fragments, and these were purified with agarose gel electrophoresis and amplified using PCR to generate libraries for sequencing using Illumina HiSeg™ 2000.

Pecan Sequence Analysis

The raw reads were quality trimmed with a custom Perl script, removing bases from the ends of each read until two consecutive bases with quality scores >=30 were reached. Read pairs with one or both reads less than 30 bp long after trimming were discarded. The trimmed reads from all six samples were pooled and de novo assembled using Trinity r2013-02-25 (trinityrnaseq.github.io) with 24 threads and 100 GB of RAM allocated to the JVM. The assembled transcripts were aligned with the black walnut transcriptome (hardwoodgenomics.org/content/black-walnut-unigene-version-120313) with BLASTX, using an E-value cutoff of 0.00001 and a minimum query alignment of 75%. Annotation was assigned to each transcript based on its walnut homolog's SWISS-PROT, PFAM, and Gene Ontology classification data. Each sample was then mapped to the de novo assembly using Tophat2 v2.0.12 (ccb.jhu.edu/software/tophat/index.shtml) with a mean read pair inner distance of 100 bp, inner distance standard deviation of 50, and a maximum intron length of 25,000. Transcript assembly and quantification was done by Cufflinks v2.2.1 with 24 threads and the default values of all other parameters. The six transcript sets identified by Cufflinks were merged using Cuffmerge v2.2.1. Differential expression testing was performed with Cuffdiff v2.2.1, treating the three Kanza samples and the three Pawnee samples as replicates.

Pecan SNP Discovery and Annotation

SNPs and indels (insertions/deletions) were called using the Samtools v0.1.18 (samtools.sourceforge.net) “mpileup” command and bcftools (part of the Samtools package). The bcftools output was reformatted and filtered with a custom Perl script, keeping only those SNPs and INDELs with total coverage >=5, reference and alternative coverages both >=2, and alternative allele frequencies of at least 10. A confidence score was generated based on the percent of nucleotides matching the surrounding regions of the location of the SNP. Low confidence regions with less than 90% identity where a SNP was called were not included in the final SNP predictions. SNPs were quality controlled by remapping the transcript reads back onto the assembly and checked against the ACE alignment files. Small indels were also identified and the process was biased towards getting good quality SNPs vs. getting all the SNPs at the expense of false SNP calls. The walnut annotation includes homologs in the SWISS-PROT database, PFAM classification, and Gene Ontology classification.

SNP Genotyping and HRM Analysis

Primer3 was used to design primers targeting candidate SNPs from the generated pecan sequences. Criteria for primer design include a predicted annealing temperature (Tm) of 59° C. to 61° C. with an optimum temperature of 60° C., primer length ranging between 18-24 bp, and PCR amplicon lengths of 40 to 200 bp. All PCR reactions for HRM were performed in 384-well plates using a 9700 Thermal Cycler (Applied Biosystems, Foster City, Calif., USA) using a total volume of 15 μL per well. HRM enables the identification of sequence variants in a target region without sequencing. HRM was successfully used for SNP genotyping and mapping in plants (Han, et al., 2012). The PCR reaction mixture consisted of 5 ng of genomic DNA, 0.25 μM of forward and reverse primer, 1× LightScanner High Sensitivity Master Mix (Idaho Technologies, Salt Lake, Utah, USA) and 10 μL mineral oil. After an initial denaturation step of 2 min at 95° C., 45 PCR cycles were performed with 30 s of denaturation at 94° C. and 30 s at the target annealing temperature of 60° C., followed by a final hold at 4° C. Samples were then transferred to a LightScanner 384-well system (Idaho Technologies, Salt Lake, Utah) and a melting cycle was performed by increasing the temperature at 0.1° C. s⁻¹ from 56 to 95° C. Melting data was analyzed and visualized with the LightScanner Software with CALL-IT 2.0 (Idaho Technologies, Salt Lake, Utah) using the small amplicon module.

Amplicon Sequencing by Sanger Method

PCR were performed at 60° C. in final volume of 20 μl using forward and reverse primers (5 μM each, 1 μl each), 2 μl 10×PCR buffer, 2 μl MgCl₂ (25 mM), 2 μl dNTP mix (2 mM), 9.80 μl of nuclease free water, 0.2 μl Promega GoTaq DNA polymerase (5U/μl) and 2 μl of DNA at 10 ng/μl. PCR products were cleaned with the ExoSAP-IT kit (Affymetrix, Cleveland, Ohio) and sequenced in a final volume of 10 μl using both forward and reverse primers (1.6 μM, 2 μl) designed for HRM and a BigDye (0.7 μl) terminator v3.1 Cycle Sequencing Kit (Thermal Fisher Inc., Foster City, Calif.), A.B.I. buffer (3.65 μl), DMSO (0.5 μl) and water until a final reaction volume of 10 μl. An ABI3730 automated sequencer (Thermal Fisher Inc., Foster City, Calif.) was used to perform the PCR reactions. Sequences were mapped and aligned using Geneious v.7.0.5 (geneious.com; Kearse, et al., 2012).

Results

SNP Discovery and Validation

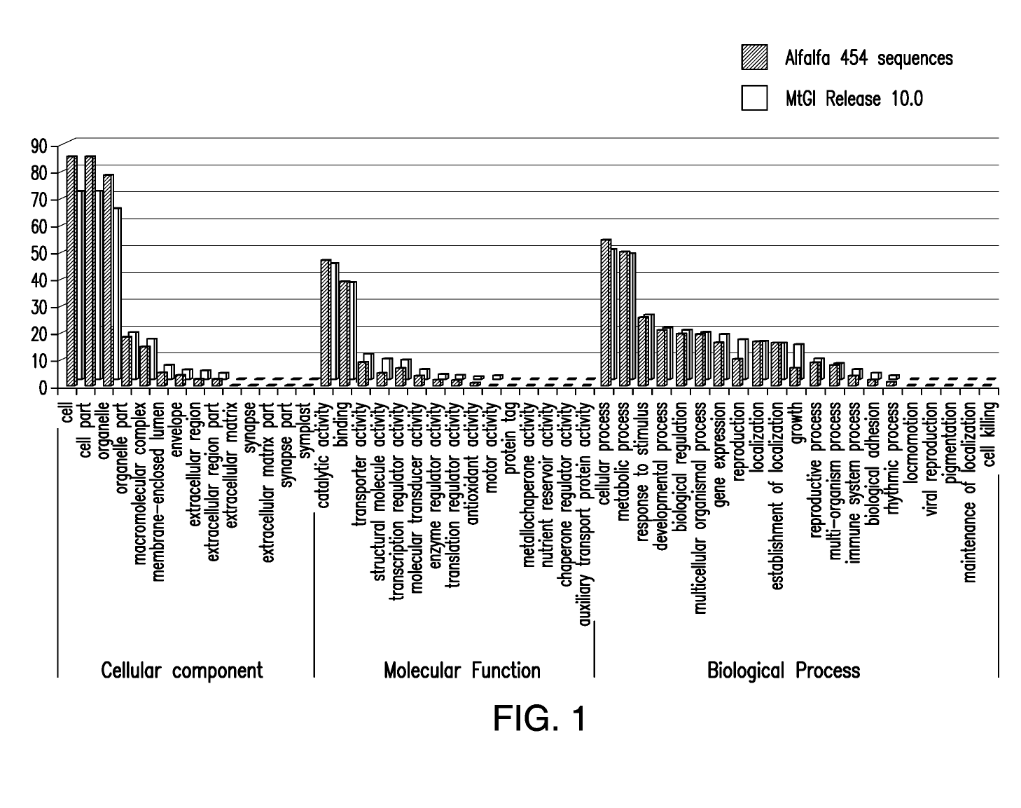

The total number of reads for the two pecan genotypes ranged from 4.63 Gb to 9.81 Gb (Table 1; Statistics of the clean sequence reads from the two pecan cultivars (Pawnee=14P38, 14P39, 14P30) and Kanza (14L18, 14K19 and 14K20)) and covered a range of functional categories based on the gene ontology assignments (FIG. 1).

TABLE 1

| | Total | % Q20 | % Q20 | % GC | % GC | | | Filter | |
|---|---|---|---|---|---|---|---|---|---|
| | Reads | Before | After | Before | After | Filter | | Low | Clean |
| SampleID | nt | Filter^{‡} | Filter | Filter | Filter | Adapter | Filter N | Quality | Reads nt |
| 14k18 | 9.81G | 98.09% | 98.94% | 45.28% | 45.25% | 1.04% | 0.12% | 1.67% | 9.53G |
| 14k19 | 6.36G | 98.16% | 98.93% | 45.14% | 45.13% | 0.49% | 0.12% | 1.61% | 6.22G |
| 14k20 | 6.25G | 98.08% | 98.93% | 45.31% | 45.28% | 1.06% | 0.12% | 1.74% | 6.07G |
| 14p38 | 4.63G | 98.08% | 98.93% | 45.39% | 45.36% | 0.63% | 0.12% | 1.73% | 4.51G |
| 14p39 | 4.65G | 98.12% | 98.95% | 45.38% | 45.37% | 0.72% | 0.12% | 1.69% | 4.53G |
| 14p40 | | | | | | | | | |

| | |
|---|---|
| Total Reads nt | Nucleotides number of total reads |
| ^{‡}% Q20 Before Filter | The number of Nucleotides with quality higher |
| | than 20/Nucleotides (Reads1, Reads2) Before Filter |
| % Q20 After Filter | The number of Nucleotides with quality higher than |
| | 20/Nucleotides (Reads1, Reads2) After Filter |
| % GC Before Filter | GC number/Nucleotides (Reads1, Reads2) Before Filter |
| % GC After Filter | GC number/Nucleotides (Reads1, Reads2) After |
| | Filter |
| Filter Adapter | Number of reads with adaptors/Total reads number |
| | (Reads1, Reads2) |
| Filter N | N number/Nucleotides (Reads1, Reads2) |
| Filter Low Quality | Number of low quality reads/Total reads number |
| | (Reads1, Reads2) |
| Clean Reads nt | Nucleotides number of Clean Reads |

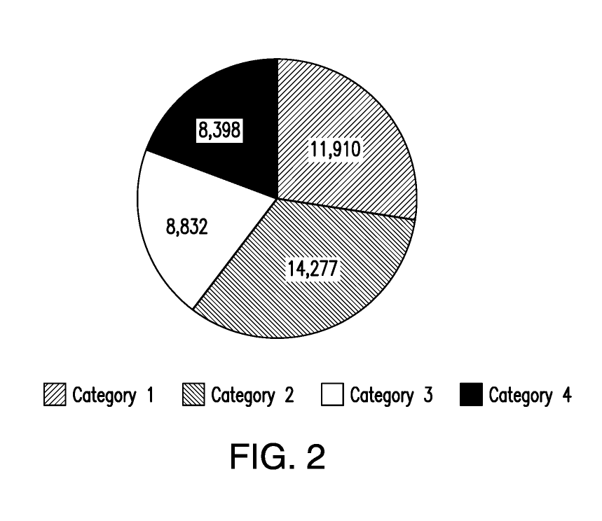

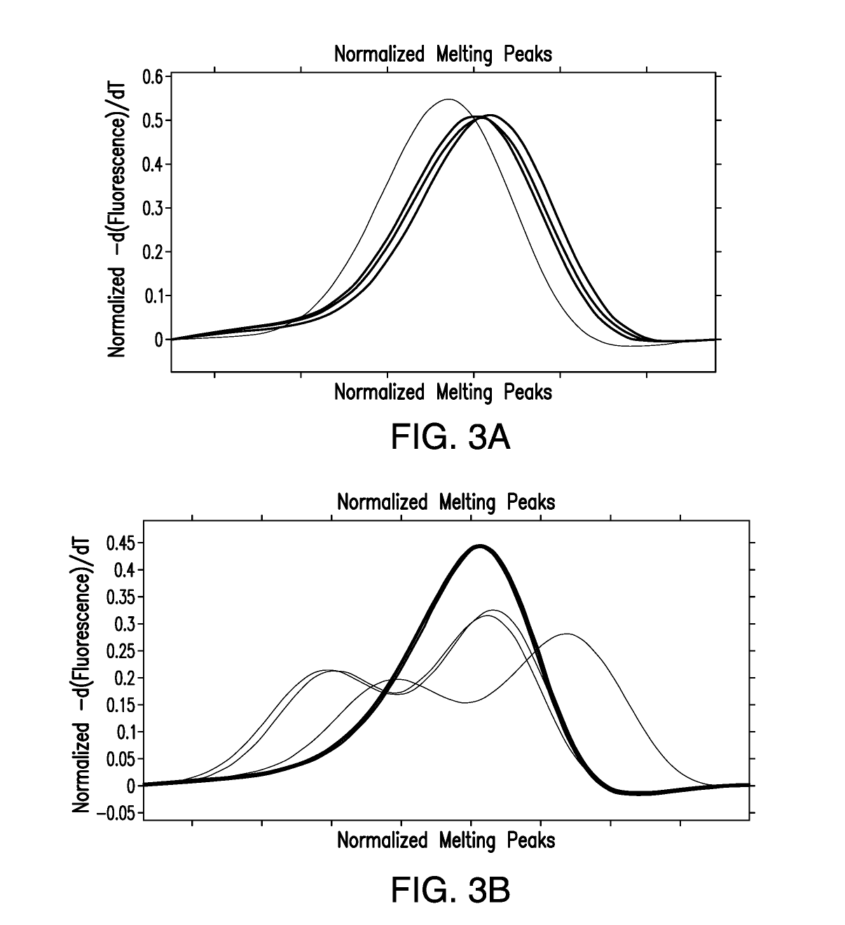

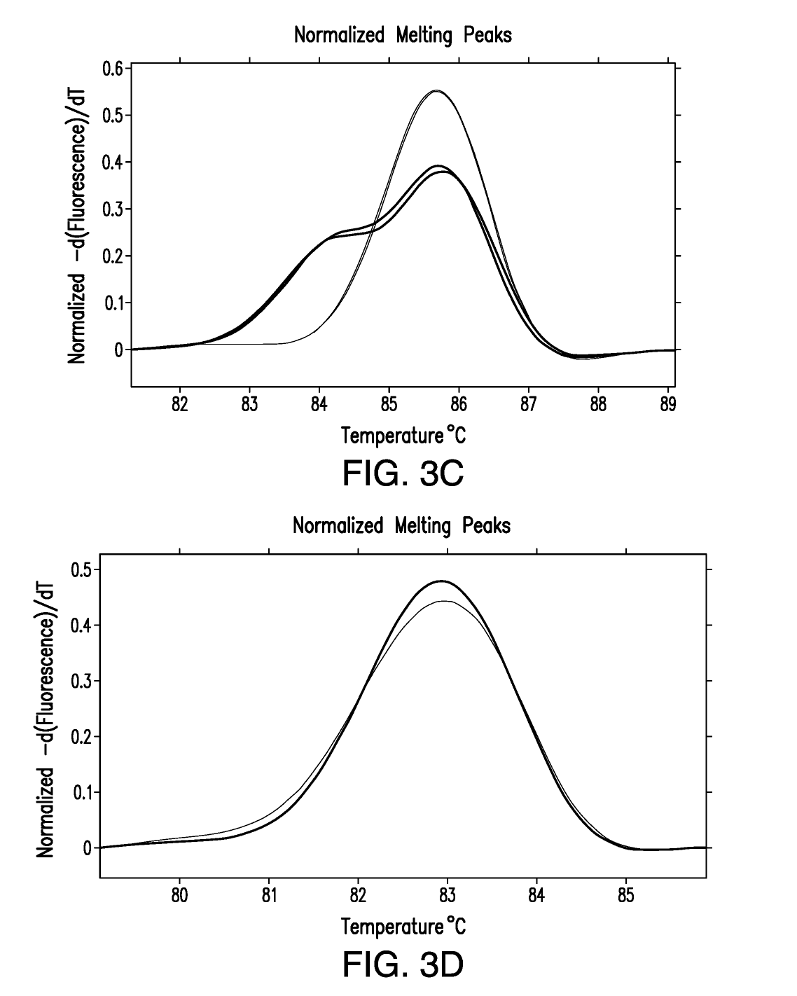

Candidate SNPs were called based on sequence alignments between the Pawnee and Kanza genotypes. A total of 43,417 candidate SNPs were identified between the two genotypes (FIG. 2). The SNPs were assigned to Categories based on their allelic composition (FIG. 2). Category 1 SNPs are between the two genotypes and homozygous within each genotype (FIG. 2). For example, CI0001 is polymorphic between Pawnee (GG) and Kanza (AA) (FIG. 3A). Category 1 is the second most abundant type with 11,910 which is 27.4% of all candidate SNPs (FIG. 3A). Category 2 SNPs are heterozygous within one genotype and homozygous in the other (FIG. 2). Category 2 SNP is the most abundant type of SNPs (14,277) and constitutes 32.9% of all candidate SNPs. A predicted Category 2 SNP, CI0030 was identified between Pawnee (TT) and Kanza (CC). The Kanza genotype appears to have more than one SNP in the target amplicon (FIG. 3B). Category 3 SNPs are biallelic and heterozygous within each genotype (e.g., Pawnee=AC and Kanza=AC). SNPs in Category 3 (FIG. 3C) are the least abundant class of SNPs and account for only 20.3% (8,832 SNPs) of all candidate SNPs identified. SNP CI0091 confirms the presence of a single SNP in Pawnee (CG) and Kanza (CC). Category 4 SNPs have more than two variants (e.g., Pawnee=A/C/G and/or Kanza=A/C/T). For simplicity and ease in future mapping efforts, SNP validation focused on Category 1, Category 2 and Category 3 SNPs. Candidate SNPs showing an overlapping melting profile in the HRM analysis for both pecan genotypes indicated the absence of a predicted SNP based on the RNA Seq data, as is the case for primer CI0094 (FIG. 3D). Candidate SNPs in other categories as well as INDELs were not considered for validation due to the complexity of their segregation patterns and potential of false positives due to lack of sufficient depths of sequencing coverage.

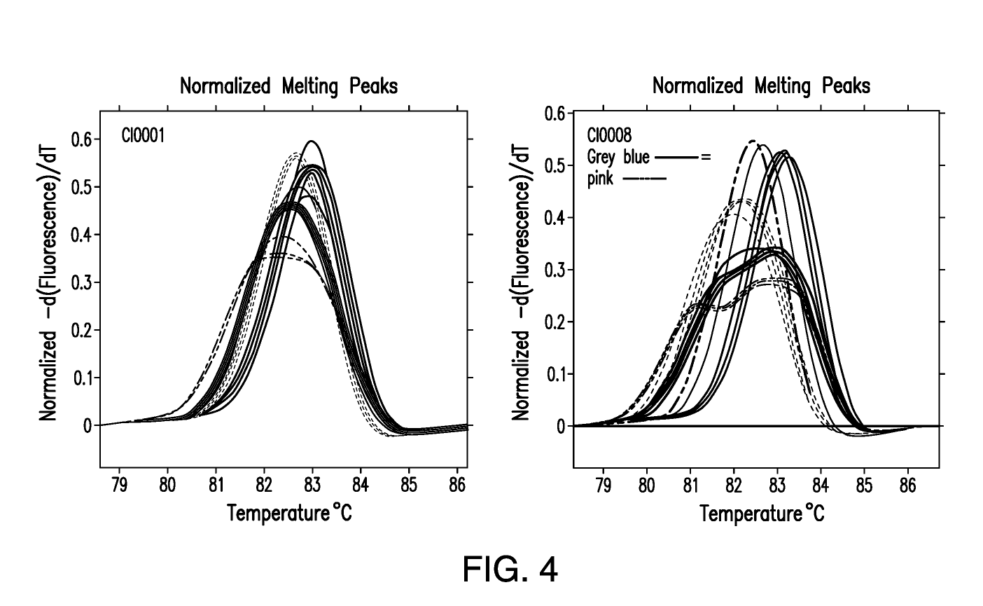

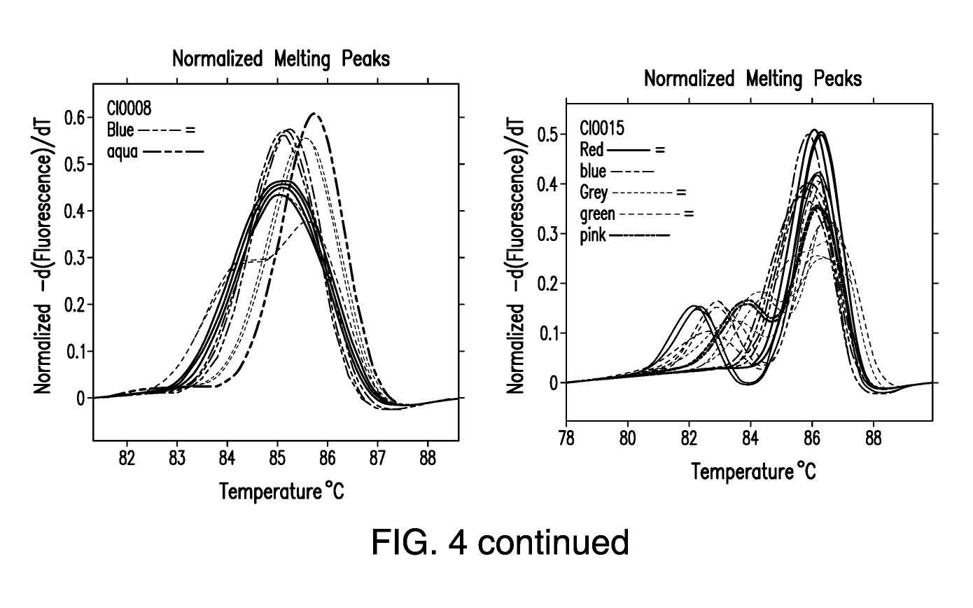

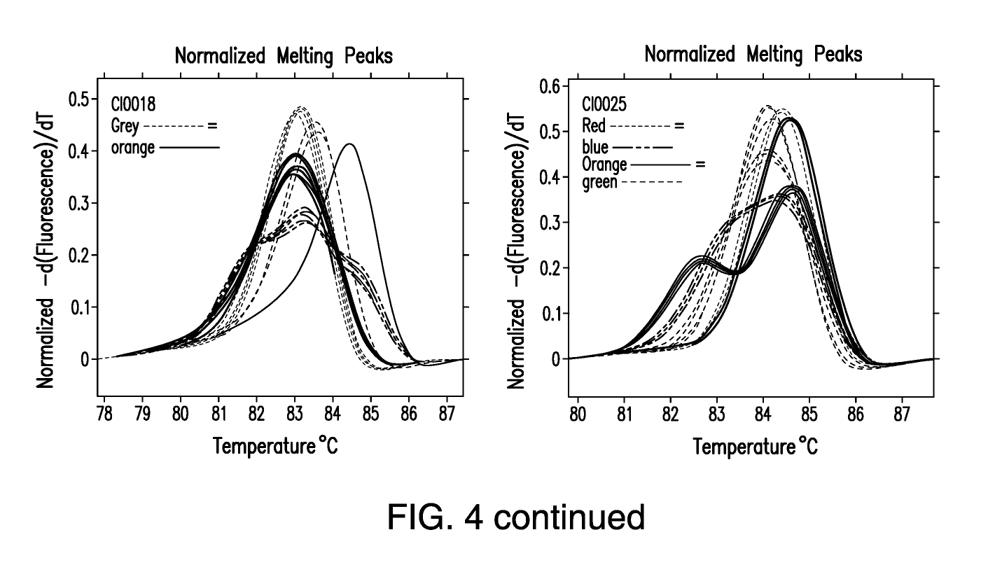

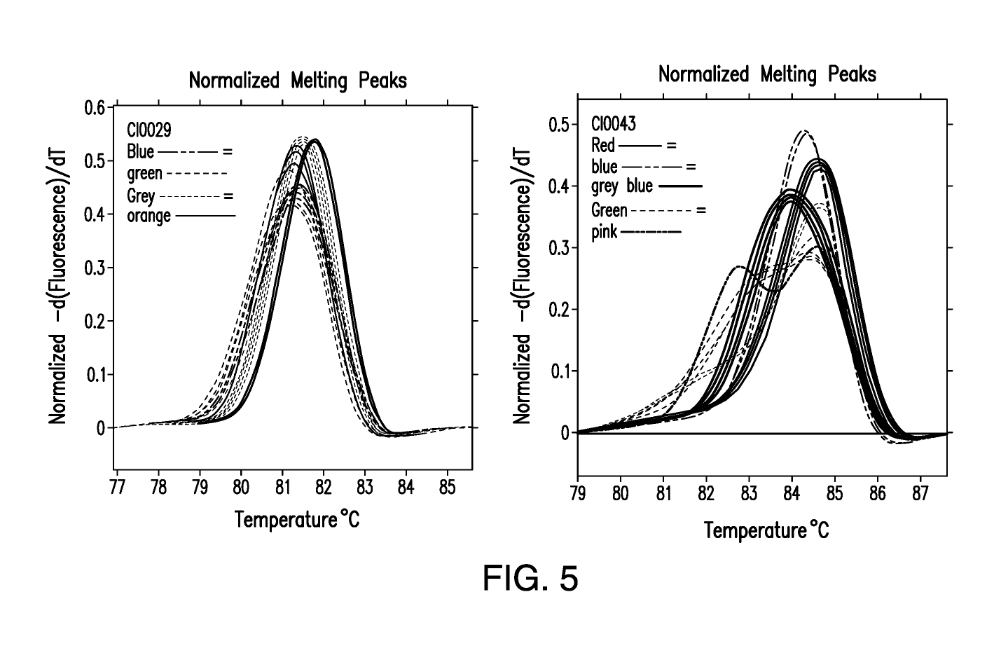

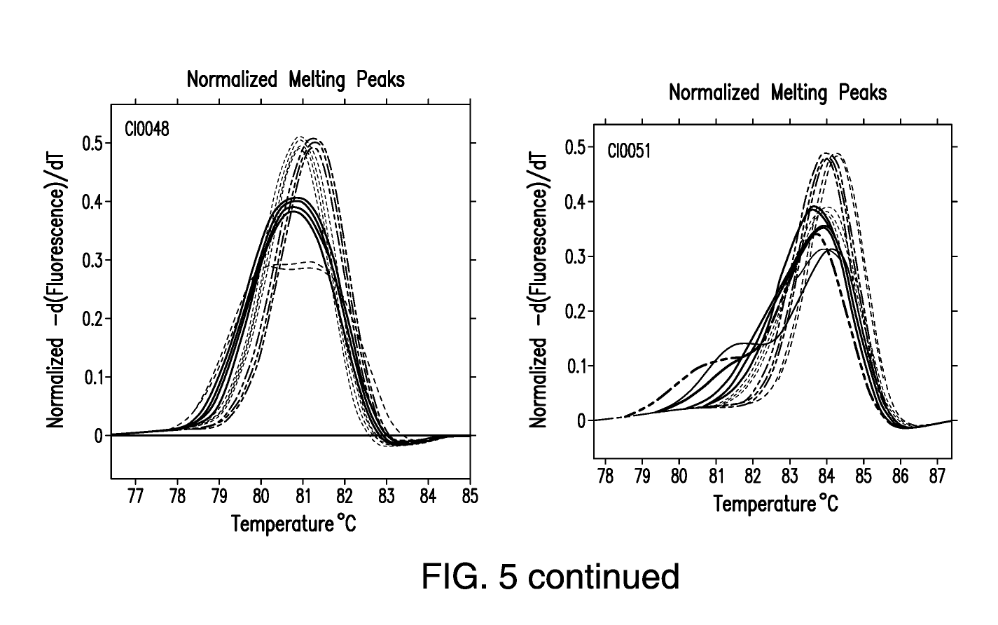

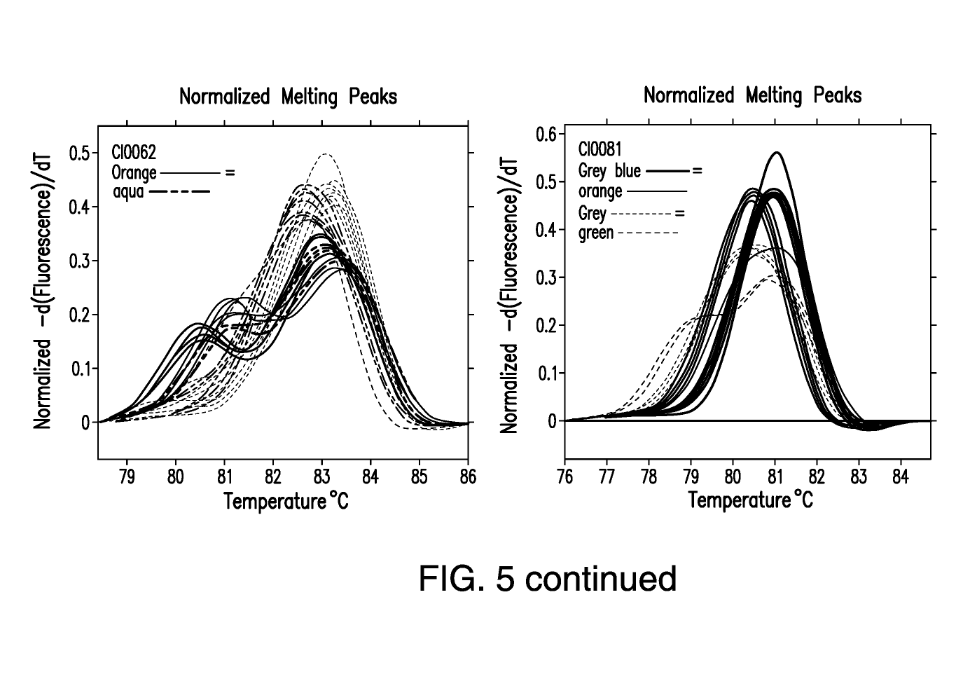

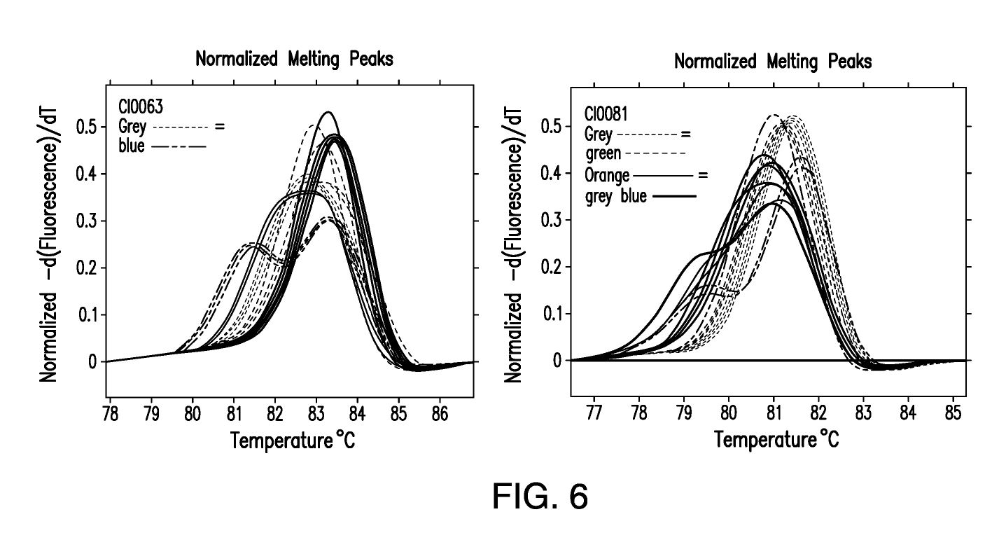

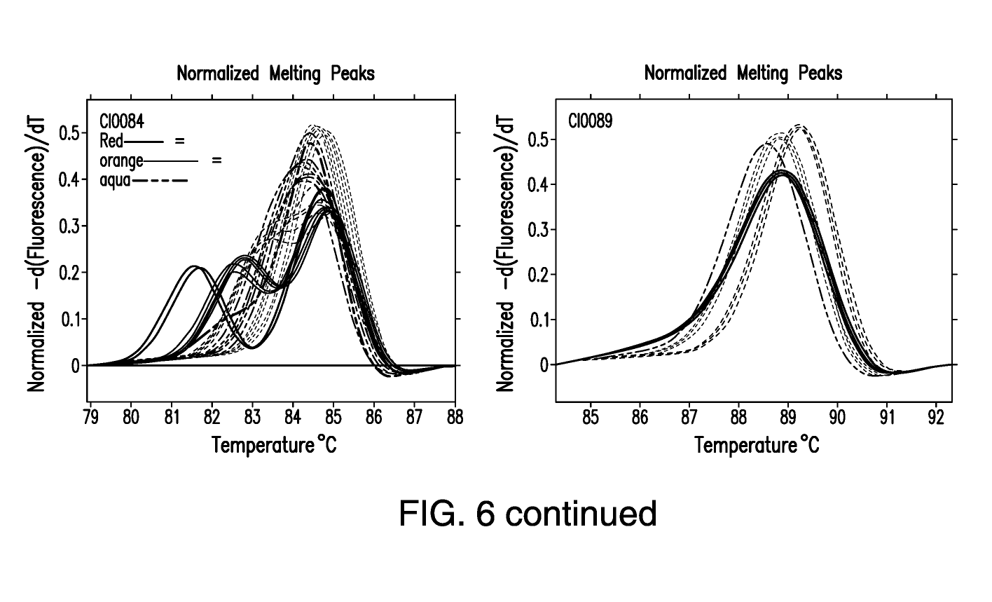

Using a set of sixteen SNP primer pairs (Table 2), sixty pecan cultivars were distinguished (from the 62 pecan ORTETs evaluated) based on the HRM profiles of the pecan ORTET samples (FIG. 4, FIG. 5 and FIG. 6), with target pecan sequence and primer information in Table 3 and data for groupings presented in Tables 4, 5 and 6.

TABLE 2

| | | SNP | Genotype | | | | Product |
|---|---|---|---|---|---|---|---|
| Transcript ID | Primer Id | Position | Kanza | Pawnee | Forward primer | Reverse primer | size |
| comp100039_c0_seq1 | CI0001 | 1387 | A/A | G/G | SEQ ID NO: 1 | SEQ ID NO: 2 | 195 |
| comp102718_c0_seq1 | CI0008B | 79 | A/A | G/G | SEQ ID NO: 3 | SEQ ID NO: 4 | 216 |
| comp102965_c0_seq1 | CI0012 | 489 | G/G | A/A | SEQ ID NO: 5 | SEQ ID NO: 6 | 176 |
| comp88992_c1_seq1 | CI0015 | 225 | C/C | T/T | SEQ ID NO: 7 | SEQ ID NO: 8 | 148 |
| comp103993_c0_seq1 | CI0018 | 212 | T/T | C/C | SEQ ID NO: 9 | SEQ ID NO: 10 | 169 |
| comp104325_c0_seq1 | CI0025 | 1653 | C/C | T/T | SEQ ID NO: 11 | SEQ ID NO: 12 | 184 |
| comp88872_c2_seq1 | CI0029 | 271 | T/T | C/C | SEQ ID NO: 13 | SEQ ID NO: 14 | 181 |
| comp88428_c0_seq1 | CI0043 | 926 | C/A | C/C | SEQ ID NO: 15 | SEQ ID NO: 16 | 152 |
| comp885763_c0_seq1 | CI0048 | 86 | T/A | T/T | SEQ ID NO: 17 | SEQ ID NO: 28 | 156 |
| comp105216_c0_seq9 | CI0051 | 877 | T/C | T/T | SEQ ID NO: 19 | SEQ ID NO: 20 | 170 |
| comp105247_c0_seq1 | CI0053 | 549 | C/T | C/C | SEQ ID NO: 21 | SEQ ID NO: 22 | 171 |
| comp107064_c1_seq7 | CI0058 | 1004 | T/C | T/T | SEQ ID NO: 23 | SEQ ID NO: 24 | 148 |
| comp83927_c0_seq2 | CI0062 | 342 | C/T | C/C | SEQ ID NO: 25 | SEQ ID NO: 26 | 156 |
| comp88616_c0_seq1 | CI0081 | 215 | G/G | G/T | SEQ ID NO: 27 | SEQ ID NO: 28 | 157 |
| comp88689_c0_seq1 | CI0084 | 353 | A/A | A/T | SEQ ID NO: 29 | SEQ ID NO: 30 | 169 |
| comp90257_c0_seq2 | CI0089 | 541 | A/A | A/G | SEQ ID NO: 31 | SEQ ID NO: 32 | 182 |

TABLE 3

| | | | Pecan | | | |
|---|---|---|---|---|---|---|
| | Putative target Gene | | ORTET | | | |
| Primer | based on black | | group- | Forward | Reverse | |
| ID | walnut annotation | | ing | primer | primer | Sequence |
| CI0001 | GO: 0042802 identical | | 4 | tgaatct | cagccat | ggctgcttctggctcattgctttttgaatatcctctaatcatg |
| | protein binding, | | | gctttat | agaaaca | gtgttccacatgaaagtattgggtctactaatttcgtcaaaaa |
| | GO: 0005515 | | | ttccacc | attcctc | ccttccgagcataagtcaagctgccaaaatctggggagatgct |
| | protein binding | | | a | a | ggaagccaggagcctgctcactgcgtttgggtctccaatgaga |
| | | | | (SEQ ID | (SEQ ID | cctgttttataaagtcga{gcatgaatctgctttatttccacc |
| | | | | NO: 1) | NO: 2) | attttt[221: A/C, C/C, C/C]aacatctttcaagcaga |
| | | | | | | cacagactctgt[251: C/T, T/T, C/C]ctacatttgat |
| | | | | | | tctatggaggtctgattttgtagtttcactgaaatggtttcca |
| | | | | | | tggaatggttgatatgtgataaaattggtataggttcataacg |
| | | | | | | agaagaagatgagaaaaatgaggaattgtttctatggctg}tg |
| | | | | | | attttattagaaa (SEQ ID NO: 33) |
| CI0008B | GO: 001602 | GO: 0055085 | 6 | aaggggc | ttcagtg | ccagttttgttaaggggcacctctaccagttagtttccatctt |
| | 1 integral | trans- | | acctcta | gccttgt | ctgtctcttcatcttctgttctccaaactgaagag[79: |
| | component | membrane | | ccagtt | gttctg | T/T, C/C, C/T, A/C][80: A/A, G/G, G/A]agcag |
| | of | transport | | (SEQ ID | (SEQ ID | at[88: T/T, C/C]tagaaaagtttaacca (SEQ ID |
| | membrane, | | | NO: 3) | NO: 4) | NO: 34) |
| CI0012 | PF03168.8: Late | | 4 | ttcatat | cgcccaa | ctcaacccttctagctagctccaaaatattaaaaatggcaggc |
| | embryogenesis | | | tggtctt | aatttgt | gctgatgattcagttccatttacacccagcaaactgtatccga |
| | abundant protein | | | tgccttg | gttctt | aaagcgatgaagagatcgccacgttcaaagaaatgaagaaaga |
| | | | | a | (SEQ ID | acgaagtggtaaatgtttcttttatgtttttgctggaattgtc |
| | | | | (SEQ ID | NO: 6) | atccagtgcgtt{ttcatattggtctttgccttgattacgctg |
| | | | | NO: 5) | | cgtagtagaattcccgatgttaaggtaaggtcgatt[251: |
| | | | | | | A/A, G/G, A/G]cggtgaagaatctcaggtataataatggc |
| | | | | | | aca[284: T/T, T/G]cttcggcatcaatatctccttcttt |
| | | | | | | taatgccactttggtcgccgaggtgaccattaagaacacaaat |
| | | | | | | ttgggcg}gtttaagttcgagaataccaccatgagtgtgctct |
| | | | | | | atggaggcatgaacgtcgttggagatacaaaaataggacatgg |
| | | | | | | gagtgtcaaagctagagacacccagaagatgatcaatcttaca |
| | | | | | | gtggaggtgagatccaata (SEQ ID NO: 35) |
| CI0015 | GO: 0009607 | | 3 | gccaaaa | gtgtgcg | gtcaaactgaactactcaggcggcagagagtaccgctcaaaca |
| | response to | | | ctcaaag | cctgttt | aaagagaaaaagaagaaagataacacgcacaaactaggcactc |
| | biotic | | | aaagagt | ttacct | gagacaactcagaacccaagagattacacagctgatcagaaaa |
| | stimulus | | | cc | (SEQ ID | aa{gccaaaactcaaagaaagagtccgcggcctctaccgatcc |
| | | | | (SEQ ID | NO: 8) | aagcagaagaagccaaaaacagaagtgatcttg[205: gatg |
| | | | | NO: 7) | | cacctatccc]agacag[212: C/T, T/T]accttcactaa |
| | | | | | | tttggcaaatcaatctctacccaaggtaaaaacaggcgcaca |
| | | | | | | c}acgcacctatatatatgtatatatatgtacacacaccacat |
| | | | | | | gcagtaacggcaaagaacaacatgaacaagaagatgctgcata |
| | | | | | | acccaagccaatatcgaaaaattcaagggaaccaaaaaacagg |
| | | | | | | agcagctgcagccatgatgataagccgctaccacaaccccgtc |
| | | | | | | ctctcagcaaaccagtgcggttcattc (+205-SEQ ID |
| | | | | | | NO: 36, −205-SEQ ID NO: 37) |
| CI0018 | no assembly or | | 4 | aatagac | tttccat | ttccattcttttctttttggggtgggaaaataatctcaaaact |
| | target gene | | | caccaag | tttgcat | tgacatggacatgctttggctttaggaggtttcactccctgtg |
| | | | | aactgac | gtactag | acagactgagtaggtttgtagtgtgaccaattggtaaact{aa |
| | | | | aaa | acc | tagaccaccaagaactgacaaagctaacacagcatttagtctc |
| | | | | (SEQ ID | (SEQ ID | atctagctgaatacagtctgagcacaaactcttgg[207: |
| | | | | NO: 9) | NO: 10) | A/A, G/A]ccag[212: A/A, G/G, A/G]acttcataat |
| | | | | | | aaaataaaaaccgaaatcctgatccctctatctgtctctcaag |
| | | | | | | ataataggtctagtacatgcaaaatggaaa}ttaaatttacaa |
| | | | | | | gacatttccaccttataacgcacgaccctttcacttgtaagct |
| | | | | | | tgcgactgatcttaataaaattatatcttcttctataggtact |
| | | | | | | gctgaaacttgaaaattgatcacttgaactaccccccgagtat |
| | | | | | | gcatttctttgtccaaggttcttcctc (SEQ ID NO: 38) |
| CI0025 | PF06025.7: Domain of | | 3 | caaaata | caaagtg | tctggcctatggtgggtaagggcagttgaggaatttctcaatg |
| | Unknown Function | | | aagatga | cttgacg | tcctacgctccagcccatatatgcatgtgcaacagaagatgtg |
| | (DUF913) | | | catggta | tcctga | gagctctagccctcattcatcactggattccacaatcgatcag |
| | | | | gagga | (SEQ ID | ggtccatacgtaaatatcgcagatggaatggtggctgcaatgt |
| | | | | (SEQ ID | NO: 12) | gcaatgag{caaaataaagatgacatggtagaggaattacctg |
| | | | | NO: 11) | | aaccgccgaagaattctgatgaagaagataataaaa[251: |
| | | | | | | A/A, A/G, G/G]ggcttttgaagcaaaacaagatgagcacc |
| | | | | | | agacagaagcaaaagatgatgtttccccggatattgcagaaat |
| | | | | | | caaaaaccaaactacctttgttcaggacgtcaagcactttg}a |
| | | | | | | tgcaatttctccagttcgaagatgcttagatctgctgtctcgg |
| | | | | | | cagaataggtgttccatatggttgcttgcttacattgctatta |
| | | | | | | tatcaagttggccattggtaggctcagctctgcaaaccgtctt |
| | | | | | | cagaaa (SEQ ID NO: 39) |
| CI0029 | PF13561.1: Enoyl- | | 3 | ctgtaga | cgatcat | agcagcctgcttacaggataggtgtggagtcaaaaatttctaa |
| | (Acyl carrier protein) | | | ttttcct | ccaattc | aactaatgatagacaggagaacatttccttagacaagtctgca |
| | red (251: C/C) | | | atcaagg | accaca | ttaaggt{ctgtagattttcctatcaagggtgaaggtcgagtt |
| | reductase | | | gtga | (SEQ ID | gttaagaatcttgttagaaatgaggagaaattaaaatcttctc |
| | | | | (SEQ ID | NO: 14) | atgattgtaatgaattggaggataggccatctgagaatgtaat |
| | | | | NO: 13) | | ttgtgtcagaattgaggagaaagtaaaatctattca[251: |
| | | | | | | C/C, T/T, C/T]gactgtggtgaattggatgatcg}accgt |
| | | | | | | ctaagaaggcaaagttggattgttctgttaaagtatttggtga |
| | | | | | | taagaaagagaagagtgtgcggaagttacctgttgatttgagt |
| | | | | | | cgtcatgatgcaaggggtgtaggaactgtaattgattttgagg |
| | | | | | | ataaatctaggcttaaacttgttaaggattctcgaggggttga |
| | | | | | | aaatggccctttgaagaagttgaagcttgatgagaagacaaag |
| | | | | | | cctttt (SEQ ID NO: 40) |
| CI0043 | PF12560.3: Protein | | 3 | tgaatat | agtccat | agagaccccggacaaccattttgataaaatttgctgaagaggt |
| | of unknown function | | | atgttaa | ttgcaag | actacgaagagaagcaaggttaggttaatgtgtgtgcctggat |
| | (DUF3745) | | | tgctctc | tttgtgg | ttattacatgtaatgtttggatcgatcgttagcaagaacgtga |
| | | | | gaacg | (SEQ ID | {tgaatatatgttaatgctctcgaacgacttgatggactctgg |
| | | | | (SEQ ID | NO: 16) | aacatttttggccattcacttgccatttctgtcactctggcca |
| | | | | NO: 15) | | atttgtgtctatcatagatttgtattgatatatact[251: |
| | | | | | | C/C, A/C, A/A]gacttcatg}ccacaaacttgcaaatgga |
| | | | | | | cttgaatgtaatttatggagggggctctcttttattttcatca |
| | | | | | | cacagccgtaggggaaaataattttcttgttctcagaaaatgt |
| | | | | | | attctttactgttctggttgatttaggtcataactagaggtgt |
| | | | | | | tatttacatggtaagtaggattttagtttcagccgaaatcaaa |
| | | | | | | agtactgatctcatagcttccatgaacttggacgaacgatcta |
| | | | | | | ctcagt (SEQ ID NO: 41) |
| CI0048 | no assembly or | | 5 | gtctgtc | ctgcatg | aagtagtctttatatctgtct{gtctgtctgtctagctcggtc |
| | target gene | | | tgtctag | caatatg | atgtttaaatcgt[56: C/C, T/T, C/T]at[59: T/T, |
| | | | | ctcggtc | ggtgat | T/G]gatgtttgatactttttcagacattgatattacctttct |
| | | | | a | (SEQ ID | ataatgtcttctacggtgctgttaatatacgaaaattaagtcc |
| | | | | (SEQ ID | NO: 18) | tatagatgatgattttatcacccatattgcatgcag}caaaca |
| | | | | NO: 17) | | tgtaaatacacgaggctctgaatgtcctagtattgttgccgtt |
| | | | | | | tctgcgtatatcttatttatttgatcatcattctcatcatcat |
| | | | | | | catatatatatatatat (SEQ ID NO: 42) |
| CI0051 | PF13966.1: zinc- | | 7 | cataatt | aggaaga | tcaccaaatgcttttccatgggaaatgaggccgctcatgtgga |
| | binding in reverse | | | attgcga | agaaacc | tacaatgcatggtaaaacagtctcttcaaatagtactctttct |
| | transcriptase | | | tatttgt | aatccag | cagggaaaattacaatgactttatcacactcacattacgtag{ |
| | | | | acagca | tc | cataattattgcgatatttgtacagcaggtacaggttgcagat |
| | | | | (SEQ ID | (SEQ ID | cggctgcaatttgttgcaagtccccttaatttcattttctttc |
| | | | | NO: 19) | NO: 20) | [215: A/A, C/C/]g[217: C/C, A/A]ttaaactacaa |
| | | | | | | atagttcaatgtagtgga[247: T/T, A/A]tga[251: |
| | | | | | | C/C, C/T, T/T, A/T, C/A]gttgcacgtgtaaactcga |
| | | | | | | aatatgactggattggtttcttcttcct}ttctatatttgcca |
| | | | | | | acttgctgagttcagattgtgcatttgttggaatattttgtac |
| | | | | | | ccttcctatgtgcccaaatggttttctttttctttttctattt |
| | | | | | | tttctcgtttctttcaatgaaatcttatcaaagaaggaaaata |
| | | | | | | atctcatttcatctgaaatttagctttaataacttcatgtgta |
| | | | | | | cctaattacttttttg (SEQ ID NO: 43) |
| CI0053 | no assembly or | | 5 | ccaggag | aagcagc | cccaacattgtgattgacatcaaaagtgaaaaatttcagtgtt |
| | target gene | | | ataagaa | catttca | tggaagggatattcttgatgatgtttagatggttctgctctta |
| | | | | gtgtggg | gatacca | agatgttaaagaatcctaattggctttagaaggaaaaaccatg |
| | | | | ata | (SEQ ID | {ccaggagataagaagtgtgggatatatttctgtgatagtttg |
| | | | | (SEQ ID | NO: 22) | attggagtatctaacacgcggcacatatataaagacaaagaaa |
| | | | | NO: 21) | | ctcctgatattcttatctttgccactat[243: C/C, T/T, |
| | | | | | | C/T]gttaaag[251: G/G, A,A, A/G]gttcaagtggtg |
| | | | | | | gatca[269: T/G, T/T]gttgttgcagtggtatctgaaat |
| | | | | | | ggctgctt}ctttagcaagagttgttaactcatcacaagcttc |
| | | | | | | tcagctgcacccagtcatatcttttactggacaccagagcagt |
| | | | | | | tgccagtcatctctgtttagtgattctagaggtcaacttatag |
| | | | | | | ggtggactggacatgtgttctggttcctttaaatatcctcaca |
| | | | | | | atttctctcaaactgttcctcttattttagtaagatc (SEQ |
| | | | | | | ID NO: 44) |
| CI0058 | GO: 001630 | GO: 0005525 | 3 | gaaggtt | accaggg | tacctcaaaagatgagaaggctgtttgactactgctttcatat |
| | 1 kinase | GTP | | ctagttt | gtgaaga | ggaatctcagactatatctggcgaggcactatattattgacta |
| | activity, | binding, | | atgctat | gagga | cgaatgattgtactctgaaggatgatacatatctctctcccat |
| | GO: 0005524 | GO: 0006614 | | tgggta | (SEQ ID | ttgttactttatttttgttacgtcatttgttacttattata{g |
| | ATP | SRP- | | (SEQ ID | NO: 24) | aaggttctagtttatgctattgggtatatacctccatatttct |
| | binding, | dependent | | NO: 23) | | tctaccatgactatatcttaataacaagttcaatag[251: |
| | | cotrans- | | | | G/G, A/A, G/A]aattctaatagcaataaaggcttctttct |
| | | lational | | | | tgtgtgtccaaccatgatttcctctcttcacccctggt}tgga |
| | | protein | | | | tcatgcatgcaaaggagtagagtttataaaaaaaaaaa (SEQ |
| | | targeting | | | | ID NO: 45) |
| | | to | | | | |
| | | membrane | | | | |
| CI0062 | GO: 0003824 | | 4 | cttgaat | gcagagg | ctgctcacactctacattgagaagtggggggaaaaagagtgga |
| | catalytic activity | | | aatcacg | aggcatc | acaatctacttttttgttacctgcatggatacttttttatgat |
| | | | | ataggca | aagaag | cctaaagcctacaaaactttgaaaataaggaaagattactgta |
| | | | | aga | (SEQ ID | aaagt{cttgaataatcacgataggcaagatcattaacttata |
| | | | | (SEQ ID | NO: 26) | cttgaaaggcttaatgtgtctgctcatgaaacatcaaaccgaa |
| | | | | NO: 25) | | caacgatacaagtgatatta[235: C/T, T/T]cagaactt |
| | | | | | | cctcggg[251: C/C, C/T]ataagcttctgtaacaagtct |
| | | | | | | tcttgatgcctcctctgc}atttgatatatcctgtactaaagc |
| | | | | | | cacagcatccttattcgatatgacattccaaagtccatcacta |
| | | | | | | gcaagaattataaaatctacaccatcaatttcttcttcctgga |
| | | | | | | tttctggctcagcaacaacatattgctttagtagtttgtctcc |
| | | | | | | aaatgcacgagaaatggcaagaacaccaccaaccctccaagtg |
| | | | | | | ccagcccaaatgat (SEQ ID NO: 46) |
| CI0081 | no assembly or | | 4 | ttttgaa | caccaac | tttgattattgcaaatttctcgaggtaagagcaacgattttga |
| | target gene | | | tctaatg | atgcaac | aatatctgggcctgccctttggctgcccagaaaaatgaacgaa |
| | | | | gtttctc | aaatctt | attgaagaatg{ttttgaatctaatggtttctctgttgttttc |
| | | | | tgttg | c | gtctttgaaaaataatgtctagataatctcgactgagatttgg |
| | | | | (SEQ ID | (SEQ ID | gattctgttttctcttatttcctcggcaaacaaacagagagtc |
| | | | | NO: 27) | NO: 28) | [215: A/A, A/C, C/C]attttcttttttctaaagaa |
| | | | | | | [236: A/C, C/C]atttgttgcatgt[250: A/C, A/A] |
| | | | | | | ggtg}tatttagcagatcaatgtgaagttttatcttcggaatg |
| | | | | | | tgcttgcgtttggtatttgatgtggaaatccatttattcttag |
| | | | | | | aattttcctctctcttttttttttttttccctctatcttgctc |
| | | | | | | atttggatatgttgcttaattattgaaatttaatctgacgtga |
| | | | | | | ttgattggtgtctaattggatatggttgctgagaaaacggaag |
| | | | | | | gg (SEQ ID NO: 47) |
| CI0084 | no assembly or | | 4 | agaagaa | tcgagag | atatttaaagaagactcaggaaaataaatattcttaagtgata |
| | target gene | | | aagaagc | gttccct | acaaagtcaatttcattcataattagctctgaccctcagagct |
| | | | | tttcttg | gacaag | ttggaaaggaggaggaaatccgactagccattgacattaggtt |
| | | | | cac | (SEQ ID | gtgacagatcg{agaagaaaagaagctttcttgcactttccgt |
| | | | | (SEQ ID | NO: 30) | gcattcaaatgatgggaaaagtcatgagtccaccactaactac |
| | | | | NO: 29) | | tttcctacaggagatgtttc[235: A/A, A/G, G/G]ccc |
| | | | | | | caactaccattc[251: A/T, T/T, A/A]gccttttaaca |
| | | | | | | agattacaccatgagatttcaaagttacttgtcagggaacctc |
| | | | | | | tcga}aggcagggcccttcaaactcacccttatagagtaaagc |
| | | | | | | ccggtcccatacaccgtaccctcaaaagtttccttatatgaaa |
| | | | | | | ctggttaaatcgttggctttgcgcatagtcattatttgccatg |
| | | | | | | gaatattgtagattgggaggaagctgtggatggagtaaatcac |
| | | | | | | ataaaatctgagctgagcttgagt (SEQ ID NO: 48) |
| CI0089 | no assembly or | | 4 | aatgtaa | caagagg | gtcctgtaaaagtaggatttcagggaatttggaatcggatttc |
| | target gene | | | ttccgtt | catgact | ttattagttttttcgttatttgtttcttagttttgggtgtata |
| | | | | cataatt | cgaaca | tatccctctctctgtacatggaacttcaactcttttcttattt |
| | | | | tttcc | (SEQ ID | gctcatctcagtttatgataagtgacaattcaattaatt{aat |
| | | | | (SEQ ID | NO: 32) | gtaattccgttcataatttttcccatatctcgatttgttgaac |
| | | | | NO: 31) | | tctcaaaagctcctcacctgtcttgcacacagaatt[251: |
| | | | | | | G/G, A/A, G/A]ccatgcccacactatcatcatgagttggc |
| | | | | | | gtaatagcctcgtgagcggcgcggcagggactgatgccaaagt |
| | | | | | | ctgaa[329: C/C, C/T]ttgttcgagtcatgcctcttg}c |
| | | | | | | gcacgtaaagattgttcccgagtggggtgggtatgtgacggtt |
| | | | | | | ttaacctgtttagtacaatacccagtgtccacctccacttcca |
| | | | | | | tgatcagatgcacgagcaagtgccgatcgactaatagttgccc |
| | | | | | | gatattatgatattattttt (SEQ ID NO: 49) |

TABLE 4

| | Primer ID | | | | | |
|---|---|---|---|---|---|---|
| | CI0001 | CI0008B | CI0012 | CI0015 | CI0018 | CI0025 |
| Putative target | GO: 0042802 | GO: 0016021 | PF03168.8: Late | GO: 0009607 | no assembly | PF06025.7: Domain |
| Gene based on | identical protein | integral | embryogenesis | response to | or target gene | of Unknown |
| black walnut | binding, GO: 00055 | component of | abundant | biotic stimulus | | Function |
| annotation | 15 protein binding | membrane, | protein | GO: 0006952 | | (DUF913) |
| | | GO: 0055085 | | defense response | | |
| | | transmembrane | | | | |
| | | transport | | | | |
| Pecan ORTET | 4 | 6 | 4 | 3 | 4 | 3 |
| grouping | | | | | | |
| Alley | red (221: C/C 251: | grey blue (79: C/T | grey (251: G/G | grey (205: no | grey (207: | orange (251: |
| | C/C) | 80: G/G 88: C/C) | 284: T/T) | insertion 212: | A/A 212: A/A) | A/G) |
| | | | | C/T) | | |
| Amling | orange (221: C/C | grey blue (79: C/T | grey (251: G/G | red (205: | grey (207: | red (251: A/A) |
| | 251: C/T) | 80: G/G 88: C/C) | 284: T/T) | insertion 212: | A/A 212: A/A) | |
| | | | | T/T) | | |
| Apache | grey (221: C/C | red (79: C/C 80: | blue (251: A/A | red (205: | blue (207: G/A | red (251: A/A) |
| | 251: T/T) | G/G 88: C/C) | 284: T/T) | insertion 212: | 212: A/A) | |
| | | | | T/T) | | |
| Barton | orange (221: C/C | blue (79: A/C 80: | red (251: A/A | grey (205: no | grey (207: | red (251: A/A) |
| | 251: C/T) | G/A 88: C/C) | 284: T/G) | insertion 212: | A/A 212: A/A) | |
| | | | | C/T) | | |
| Brooks | orange (221: C/C | grey blue (79: C/T | red (251: A/A | orange | grey (207: | orange (251: |
| | 251: C/T) | 80: G/G 88: C/C) | 284: T/G) | | A/A 212: A/A) | A/G) |
| Burkett | grey (221: C/C | grey blue (79: C/T | blue (251: A/A | orange | grey (212: A/A | orange (251: |
| | 251: T/T) | 80: G/G 88: C/C) | 284: T/T) | | 217: A/A) | A/G) |
| Byrd | orange (221: C/C | red (79: C/C 80: | red (251: A/A | red (205: | green (207: | red (251: A/A) |
| | 251: C/T) | G/G 88: C/C) | 284: T/G) | insertion 212: | A/A 212: G/G) | |
| | | | | T/T) | | |
| Caddo | grey (221: C/C | grey blue (79: C/T | green (251: A/G | red (205: | grey (207: | orange (251: |
| | 251: T/T) | 80: G/G 88: C/C) | 284: T/G) | insertion 212: | A/A 212: A/A) | A/G) |
| | | | | T/T) | | |
| Candy | red (221: C/C 251: | grey blue (79: C/T | grey (251: G/G | red (205: | grey (207: | grey (251: G/G) |
| | C/C) | 80: G/G 88: C/C) | 284: T/T) | insertion 212: | A/A 212: A/A) | |
| | | | | T/T) | | |
| Cape Fear | orange (221: C/C | red (79: C/C 80: | red (251: A/A | grey (205: no | grey (207: | red (251: A/A) |
| | 251: C/T) | G/G 88: C/C) | 284: T/G) | insertion 212: | A/A 212: A/A) | |
| | | | | C/T) | | |
| Carmichael | red (221: C/C 251: | grey blue (79: C/T | red (251: A/A | grey (205: no | grey (207: | orange (251: |
| | C/C) | 80: G/G 88: C/C) | 284: T/G) | insertion 212: | A/A 212: A/A) | A/G) |
| | | | | C/T) | | |
| Carter | grey (221: C/C | green (79: T/T 80: | grey (251: G/G | grey (205: no | grey (207: | red (251: A/A) |
| | 251: T/T) | G/A 88: T/T) | 284: T/T) | insertion 212: | A/A 212: A/A) | |
| | | | | C/T) | | |
| Cheyenne | grey (221: C/C | grey blue (79: C/T | red (251: A/A | grey (205: no | grey (207: | red (251: A/A) |
| | 251: T/T) | 80: G/G 88: C/C) | 284: T/G) | insertion 212: | A/A 212: A/A) | |
| | | | | C/T) | | |
| Choctaw | orange (221: C/C | blue (79: A/C 80: | red (251: A/A | red (205: | grey (207: | red (251: A/A) |
| | 251: C/T) | G/A 88: C/C) | 284: T/G) | insertion 212: | A/A 212: A/A) | |
| | | | | T/T) | | |
| Clark | grey (221: C/C | grey blue (79: C/T | green (251: A/G | grey (205: no | grey (207: | orange (251: |
| | 251: T/T) | 80: G/G 88: C/C) | 284: T/G) | insertion 212: | A/A 212: A/A) | A/G) |
| | | | | C/T) | | |
| Creek | red (221: C/C 251: | blue (79: A/C 80: | blue (251: A/A | red (205: | red (208: A/A | orange (251: |
| | C/C) | G/A 88: C/C) | 284: T/T) | insertion 212: | 212: A/G) | A/G |
| | | | | T/T) | | |
| Curtis | orange (221: C/C | grey blue (79: C/T | red (251: A/A | red (205: | blue (207: G/A | orange (251: |
| | 251: C/T) | 80: G/G 88: C/C) | 284: T/G) | insertion 212: | 212: A/A) | A/G) |
| | | | | T/T) | | |
| Dependable 6-2 | orange (221: C/C | blue (79: A/C 80: | red (251: A/A | red (205: | red (208: A/A | red (251: A/A) |
| | 251: C/T) | G/A 88: C/C) | 284: T/G) | insertion 212: | 212: A/G) | |
| | | | | T/T) | | |
| Dependable 8-3 | orange (221: C/C | blue (79: A/C 80: | red (251: A/A | red (205: | red (208: A/A | red (251: A/A) |
| | 251: C/T) | G/A 88: C/C) | 284: T/G) | insertion 212: | 212: A/G) | |
| | | | | T/T) | | |
| Desirable 18-18 | orange (221: C/C | green (79: T/T 80: | blue (251: A/A | grey (205: no | grey (207: | red (251: A/A) |
| | 251: C/T) | G/A 88: T/T) | 284: T/T) | insertion 212: | A/A 212: A/A) | |
| | | | | C/T) | | |
| Desirable 5-2 | orange (221: C/C | green (79: T/T 80: | red (251: A/A | grey (205: no | grey (207: | red (251: A/A) |
| | 251: C/T) | G/A 88: T/T) | 284: T/T) | insertion 212: | A/A 212: A/A) | |
| | | | | C/T) | | |
| Eclipse | red (221: C/C 251: | grey blue (79: C/T | red (251: A/A | red (205: | red (208: A/A | orange (251: |
| | C/C) | 80: G/G 88: C/C) | 284: T/G) | insertion 212: | 212: A/G) | A/G) |
| | | | | T/T) | | |
| Elliott | orange (221: C/C | red (79: C/C 80: | red (251: A/A | red (205: | red (208: A/A | red (251: A/A) |
| | 251: C/T) | G/G 88: C/C) | 284: T/G) | insertion 212: | 212: A/G) | |
| | | | | T/T) | | |
| Evers | grey (221: C/C | grey blue (79: C/T | red (251: A/A | grey (252: C/T | grey (207: | orange (251: |
| | 251: T/T) | 80: G/G 88: C/C) | 284: T/G) | 259: insertion) | A/A 212: A/A) | A/G) |
| Excel | orange (221: C/C | blue (79: A/C 80: | red (251: A/A | grey blue (205: | blue (207: G/A | red (251: A/A) |
| | 251: C/T) | G/A 88: C/C) | 284: T/G) | insertion 212: | 212: A/A) | |
| | | | | C/T) | | |
| Forkert | grey (221: C/C | blue (79: A/C 80: | red (251: A/A | red (205: | grey (207: | red (251: A/A) |
| | 251: T/T) | G/A 88: C/C) | 284: T/G) | insertion 212: | A/A 212: A/A) | |
| | | | | T/T) | | |
| Gafford | orange (221: C/C | blue (79: A/C 80: | blue (251: A/A | red (205: | grey (207: | orange (251: |
| | 251: C/T) | G/A 88: C/C) | 284: T/T) | insertion 212: | A/A 212: A/A) | A/G) |
| | | | | T/T) | | |
| Giles | red (221: C/C 251: | grey blue (79: C/T | blue (251: A/A | grey (205: no | blue (207: G/A | orange (251: |
| | C/C) | 80: G/G 88: C/C) | 284: T/T) | insertion 212: | 212: A/A) | A/G) |
| | | | | C/T) | | |
| Halbert | red (221: C/C 251: | red (79: C/C 80: | red (251: A/A | red (205: | grey (207: | red (251: A/A) |
| | C/C) | G/G 88: C/C) | 284: T/G) | insertion 212: | A/A 212: A/A) | |
| | | | | T/T) | | |
| Humble | red (221: C/C 251: | red (79: C/C 80: | blue (251: A/A | grey (205: no | red (208: A/A | orange (251: |
| | C/C) | G/G 88: C/C) | 284: T/T) | insertion 212: | 212: A/G) | A/G) |
| | | | | C/T) | | |
| Jackson BW | grey (221: C/C | grey blue (79: C/T | blue (251: A/A | grey (205: no | grey (207: | orange (251: |
| | 251: T/T) | 80: G/G 88: C/C) | 284: T/T) | insertion 212: | A/A 212: A/A) | A/G) |
| | | | | C/T) | | |
| Jackson LA | blue (221: A/C | green (79: T/T | blue (251: A/A | grey (205: no | red (208: A/A | red (251: A/A) |
| | 251: C/T) | 80: G/A 88: T/T) | 284: T/T) | insertion 212: | 212: A/G) | |
| | | | | C/T) | | |
| Jenkins | orange (221: C/C | green (79: T/T 80: | red (251: A/A | red (205: | red (208: A/A | red (251: A/A) |
| | 251: C/T) | G/A 88: T/T) | 284: T/G) | insertion 212: | 212: A/G) | |
| | | | | T/T) | | |
| Kanza | grey (221: C/C | grey blue (79: C/T | grey (251: G/G | grey (205: no | grey (207: | grey (251: G/G) |
| | 251: T/T) | 80: G/G 88: C/C) | 284: T/T) | insertion 212: | A/A 212: A/A) | |
| | | | | C/T) | | |
| Kiowa | orange (221: C/C | grey blue (79: C/T | blue (251: A/A | grey (205: no | grey (207: | red (251: A/A) |
| | 251: C/T) | 80: G/G 88: C/C) | 284: T/T) | insertion 212: | A/A 212: A/A) | |
| | | | | C/T) | | |
| Lakota | orange (221: C/C | red (79: C/C 80: | red (251: A/A | grey (205: no | grey (207: | orange (251: |
| | 251: C/T) | G/G 88: C/C) | 284: T/G) | insertion 212: | A/A 212: A/A) | A/G) |
| | | | | C/T) | | |
| Mahan | red (221: C/C 251: | Negative | blue (251: A/A | red (205: | red (208: A/A | red (251: A/A) |
| | C/C) | | 284: T/T) | insertion 212: | 212: A/G) | |
| | | | | T/T) | | |
| Major | grey (221: C/C | red (79: C/C 80: | grey (251: G/G | grey (205: no | grey (207: | orange (251: |
| | 251: T/T) | G/G 88: C/C) | 284: T/T) | insertion 212: | A/A 212: A/A) | A/G) |
| | | | | C/T) | | |
| Mohawk | orange (221: C/C | blue (79: A/C 80: | blue (251: A/A | red (205: | red (208: A/A | red (251: A/A) |
| | 251: C/T) | G/A 88: C/C) | 284: T/T) | insertion 212: | 212: A/G) | |
| | | | | T/T) | | |
| Moreland | grey (221: C/C | red (79: C/C 80: | red (251: A/A | grey (205: no | red (208: A/A | red (251: A/A) |
| | 251: T/T) | G/G 88: C/C) | 284: T/G) | insertion 212: | 212: A/G) | |
| | | | | C/T) | | |
| Nacono | grey (221: C/C | grey blue (79: C/T | red (251: A/A | grey (205: no | grey (207: | red (251: A/A) |
| | 251: T/T) | 80: G/G 88: C/C) | 284: T/G) | insertion 212: | A/A 212: A/A) | |
| | | | | C/T) | | |
| Oconee | orange (221: C/C | blue (79: A/C 80: | grey (251: G/G | red (205: | grey (207: | red (251: A/A) |
| | 251: C/T) | G/A 88: C/C) | 284: T/T) | insertion 212: | A/A 212: A/A) | |
| | | | | T/T) | | |
| Odom | grey (221: C/C | green (79: T/T 80: | red (251: A/A | grey (205: no | grey (207: | red (251: A/A) |
| | 251: T/T) | G/A 88: T/T) | 284: T/G) | insertion 212: | A/A 212: A/A) | |
| | | | | C/T) | | |
| Pawnee | red (221: C/C 251: | red (79: C/C 80: | red (251: A/A | red (205: | red (208: A/A | red (251: A/A) |
| | C/C) | G/G 88: C/C) | 284: T/G) | insertion 212: | 212: A/G) | |
| | | | | T/T) | | |
| Philema | orange (221: C/C | green (79: T/T 80: | blue (251: A/A | grey (205: no | grey (207: | red (251: A/A) |
| | 251: C/T) | G/A 88: T/T) | 284: T/T) | insertion 212: | A/A 212: A/A) | |
| | | | | C/T) | | |
| Podsednik | orange (221: C/C | Negative | blue (251: A/A | orange | blue (207: G/A | orange (251: |
| | 251: C/T) | | 284: T/T) | | 212: A/A) | A/G) |
| Riverside | orange (221: C/C | grey blue (79: C/T | red (251: A/A | red (205: | grey (207: | red (251: A/A) |
| | 251: C/T) | 80: G/G 88: C/C) | 284: T/G) | insertion 212: | A/A 212: A/A) | |
| | | | | T/T) | | |
| Russell | orange (221: C/C | orange (79: T/T 80: | blue (251: A/A | red (205: | grey (207: | red (251: A/A) |
| | 251: C/T) | G/G 88: T/T) | 284: T/T) | insertion 212: | A/A 212: A/A) | |
| | | | | T/T) | | |
| Schley | grey (221: C/C | red (79: C/C 80: | red (251: A/A | red (205: | grey (207: | red (251: A/A) |
| | 251: T/T) | G/G 88: C/C) | 284: T/G) | insertion 212: | A/A 212: A/A) | |
| | | | | T/T) | | |
| Shawnee | grey (221: C/C | red (79: C/C 80: | red (251: A/A | grey (205: no | grey (207: | red (251: A/A) |
| | 251: T/T) | G/G 88: C/C) | 284: T/G) | insertion 212: | A/A 212: A/A) | |
| | | | | C/T) | | |
| SHG | orange (221: C/C | grey blue (79: C/T | green (251: A/G | red (205: | grey (207: | orange (251: |
| | 251: C/T) | 80: G/G 88: C/C) | 284: T/G) | insertion 212: | A/A 212: A/A) | A/G) |
| | | | | T/T) | | |
| Shoshoni | grey (221: C/C | orange (79: T/T 80: | red (251: A/A | red (205: | grey (207: | orange (251: |
| | 251: T/T) | G/G 88: T/T) | 284: T/G) | insertion 212: | A/A 212: A/A) | A/G) |
| | | | | T/T) | | |
| Sioux | orange (221: C/C | grey blue (79: C/T | red (251: A/A | red (205: | grey (207: | red (251: A/A) |
| | 251: C/T) | 80: G/G 88: C/C) | 284: T/G) | insertion 212: | A/A 212: A/A) | |
| | | | | T/T) | | |
| Stuart | blue (221: A/C | grey blue (79: C/T | red (251: A/A | grey (205: no | green (207: | orange (251: |
| | 251: C/T) | 80: G/G 88: C/C) | 284: T/G) | insertion 212: | A/A 212: G/G) | A/G) |
| | | | | C/T) | | |
| Success | orange (221: C/C | aqua (79: T/T 80: | red (251: A/A | red (205: | grey (207: | red (251: A/A) |
| | 251: C/T) | A/A 88: T/T) | 284: T/G) | insertion 212: | A/A 212: A/A) | |
| | | | | T/T) | | |
| Sumner | grey (221: C/C | Negative | red (251: A/A | red (205: | grey (207: | orange (251: |
| | 251: T/T) | | 284: T/G) | insertion 212: | A/A 212: A/A) | A/G) |
| | | | | T/T) | | |
| Syrup Mill | orange (221: C/C | orange (79: T/T 80: | red (251: A/A | grey (205: no | red (208: A/A | grey (251: G/G) |
| | 251: C/T) | G/G 88: T/T) | 284: T/G) | insertion 212: | 212: A/G) | |
| | | | | C/T) | | |
| VC168 | grey (221: C/C | blue (79: A/C 80: | red (251: A/A | grey (205: no | grey (207: | red (251: A/A) |
| | 251: T/T) | G/A 88: C/C) | 284: T/G) | insertion 212: | A/A 212: A/A) | |
| | | | | C/T) | | |
| W. Schley | red (221: C/C 251: | Negative | red (251: A/A | orange | grey (207: | orange (251: |
| | C/C) | | 284: T/G) | | A/A 212: A/A) | A/G) |
| Wichita | orange (221: C/C | red (79: C/C 80: | red (251: A/A | red (205: | red (208: A/A | red (251: A/A) |
| | 251: C/T) | G/G 88: C/C) | 284: T/G) | insertion 212: | 212: A/G) | |
| | | | | T/T) | | |
| Witte | blue (221: A/C | blue (79: A/C 80: | red (251: A/A | red (205: | grey (207: | grey (251: G/G) |
| | 251: C/T) | G/A 88: C/C) | 284: T/G) | insertion 212: | A/A 212: A/A) | |
| | | | | T/T) | | |
| Zinner | grey (221: C/C | grey blue (79: C/T | red (251: A/A | grey (205: no | red (208: A/A | red (251: A/A) |
| | 251: T/T) | 80: G/G 88: C/C) | 284: T/G) | insertion 212: | 212: A/G) | |
| | | | | C/T) | | |

TABLE 5

| | Primer ID | | | | |
|---|---|---|---|---|---|
| | CI0029 | CI0043 | CI0048 | CI0051 | CI0053 |
| Putative target | PF13561.1: Enoyl- | PF12560.3: Protein | no assembly or | PF13966.1: zinc- | no assembly or |
| Gene based on | (Acyl carrier | of unknown | target gene | binding in | target gene |
| black walnut | protein) | function | | reverse | |
| annotation | reductase | (DUF3745) | | transcriptase | |
| Pecan ORTET | 3 | 3 | 5 | 7 | 5 |
| grouping | | | | | |
| Alley | blue (251: C/T) | red (251: C/C) | grey (56: T/T 59: | green (215: A/A | blue (243: T/T |
| | | | T/T) | 217: C/C 247: | 251: A/A 269: |
| | | | | T/T 251: C/C) | C/T) |
| Amling | grey (251: T/T) | red (251: C/C) | grey (56: T/T 59: | blue (215: A/A | grey (243: T/T |
| | | | T/T) | 217: C/C 247: | 251: A/A 269: |
| | | | | T/T 251: T/T) | T/T) |
| Apache | blue (251: C/T) | red (251: C/C) | blue (56: T/T 59: | blue (215: A/A | orange |
| | | | C/C) | 217: C/C 247: | (243: T/G 251: |
| | | | | T/T 251: T/T) | G/G 269: C/C) |
| Barton | grey (251: T/T) | red (251: C/C) | blue (56: T/T 59: | blue (215: A/A | blue (243: T/T |
| | | | C/C) | 217: C/C 247: | 251: A/A 269: |
| | | | | T/T 251: T/T) | C/T) |
| Brooks | grey (251: T/T) | red (251: C/C) | red (56: T/T 59: | blue (215: A/A | grey (243: T/T |
| | | | C/T) | 217: C/C 247: | 251: A/A 269: |
| | | | | T/T 251: T/T) | T/T) |
| Burkett | grey (251: T/T) | red (251: C/C) | red (56: T/T 59: | blue (215: A/A | grey (243: T/T |
| | | | C/T) | 217: C/C 247: | 251: A/A 269: |
| | | | | T/T 251: T/T) | T/T) |
| Byrd | grey (251: T/T) | red (251: C/C) | red (56: T/T 59: | red (215: A/A | red (243: T/G |
| | | | C/T) | 217: A/A 247: | 251: G/G 269: |
| | | | | T/T 251: T/T) | C/C) |
| Caddo | grey (251: T/T) | red (251: C/C) | red (56: T/T 59: | blue (215: A/A | orange |
| | | | C/T) | 217: C/C 247: | (243: T/G 251: |
| | | | | T/T 251: T/T) | G/G 269: C/C) |
| Candy | grey (251: T/T) | red (251: C/C) | grey (56: T/T 59: | green (215: A/A | grey (243: T/T |
| | | | T/T) | 217: C/C 247: | 251: A/A 269: |
| | | | | T/T 251: C/C) | T/T) |
| Cape Fear | blue (251: C/T) | green (251: A/A) | red (56: T/T 59: | blue (215: A/A | orange |
| | | | C/T) | 217: C/C 247: | (243: T/G 251: |
| | | | | T/T 251: T/T) | G/G 269: C/C) |
| Carmichael | grey (251: T/T) | red (251: C/C) | red (56: T/T 59: | blue (215: A/A | grey (243: T/T |
| | | | C/T) | 217: C/C 247: | 251: A/A 269: |
| | | | | T/T 251: T/T) | T/T) |
| Carter | grey (251: T/T) | red (251: C/C) | blue (56: T/T 59: | grey (215: A/A | blue (243: T/T |
| | | | C/C) | 217: C/C 247: | 251: A/A 269: |
| | | | | T/T 251: C/T) | C/T) |
| Cheyenne | blue (251: C/T) | red (251: C/C) | grey (56: T/T 59: | blue (215: A/A | blue (243: T/T |
| | | | T/T) | 217: C/C 247: | 251: A/A 269: |
| | | | | T/T 251: T/T) | C/T) |
| Choctaw | grey (251: T/T) | red (251: C/C) | grey (56: T/T 59: | red (215: A/A | grey (243: T/T |
| | | | T/T) | 217: A/A 247: | 251: A/A 269: |
| | | | | T/T 251: T/T) | T/T) |
| Clark | red (251: C/C) | red (251: C/C) | grey (56: T/T 59: | grey (215: A/A | grey (243: T/T |
| | | | T/T) | 217: C/C 247: | 251: A/A 269: |
| | | | | T/T 251: C/T) | T/T) |
| Creek | grey (251: T/T) | green (251: A/A) | blue (56: T/T 59: | grey (215: A/A | grey (243: T/T |
| | | | C/C) | 217: C/C 247: | 251: A/A 269: |
| | | | | T/T 251: C/T) | T/T) |
| Curtis | blue (251: C/T) | grey (251: A/C) | orange (56: T/G | blue (215: A/A | grey (243: T/T |
| | | | 59: C/C) | 217: C/C 247: | 251: A/A 269: |
| | | | | T/T 251: T/T) | T/T) |
| Dependable 6-2 | grey (251: T/T) | red (251: C/C) | Negative | grey (215: A/A | grey (243: T/T |
| | | | | 217: C/C 247: | 251: A/A 269: |
| | | | | T/T 251: C/T) | T/T) |
| Dependable 8-3 | grey (251: T/T) | red (251: C/C) | blue (56: T/T 59: | grey (215: A/A | grey (243: T/T |
| | | | C/C) | 217: C/C 247: | 251: A/A 269: |
| | | | | T/T 251: C/T) | T/T) |
| Desirable 18-18 | grey (251: T/T) | red (251: C/C) | grey (56: T/T 59: | grey (215: A/A | blue (243: T/T |
| | | | T/T) | 217: C/C 247: | 251: A/A 269: |
| | | | | T/T 251: C/T) | C/T) |
| Desirable 5-2 | grey (251: T/T) | red (251: C/C) | grey (56: T/T 59: | grey (215: A/A | blue (243: T/T |
| | | | T/T) | 217: C/C 247: | 251: A/A 269: |
| | | | | T/T 251: C/T) | C/T) |
| Eclipse | red (251: C/C) | red (251: C/C) | red (56: T/T 59: | blue (215: A/A | red (243: T/G |
| | | | C/T) | 217: C/C 247: | 251: G/G 269: |
| | | | | T/T 251: T/T) | C/C) |
| Elliott | grey (251: T/T) | grey (251: A/C) | green (56: T/G | red (215: A/A | grey (243: T/T |
| | | | 59: C/T) | 217: A/A 247: | 251: A/A 269: |
| | | | | T/T 251: T/T) | T/T) |
| Evers | grey (251: T/T) | Negative | Negative | grey (215: A/A | grey (243: T/T |
| | | | | 217: C/C 247: | 251: A/A 269: |
| | | | | T/T 251: C/T) | T/T) |
| Excel | grey (251: T/T) | red (251: C/C) | grey (56: T/T 59: | blue (215: A/A | grey (243: T/T |
| | | | T/T) | 217: C/C 247: | 251: A/A 269: |
| | | | | T/T 251: T/T) | T/T) |
| Forkert | grey (251: T/T) | red (251: C/C) | red (56: T/T 59: | red (215: A/A | orange |
| | | | C/T) | 217: A/A 247: | (243: T/G 251: |
| | | | | T/T 251: T/T) | G/G 269: C/C) |
| Gafford | grey (251: T/T) | red (251: C/C) | blue (56: T/T 59: | blue (215: A/A | blue (243: T/T |
| | | | C/C) | 217: C/C 247: | 251: A/A 269: |
| | | | | T/T 251: T/T) | C/T) |
| Giles | grey (251: T/T) | green (251: A/A) | red (56: T/T 59: | red (215: A/A | grey (243: T/T |
| | | | C/T) | 217: A/A 247: | 251: A/A 269: |
| | | | | T/T 251: T/T) | T/T) |
| Halbert | grey (251: T/T) | red (251: C/C) | red (56: T/T 59: | blue (215: A/A | grey (243: T/T |
| | | | C/T) | 217: C/C 247: | 251: A/A 269: |
| | | | | T/T 251: T/T) | T/T) |
| Humble | grey (251: T/T) | red (251: C/C) | grey (56: T/T 59: | grey (215: A/A | blue (243: T/T |
| | | | T/T) | 217: C/C 247: | 251: A/A 269: |
| | | | | T/T 251: C/T) | C/T) |
| Jackson BW | blue (251: C/T) | red (251: C/C) | red (56: T/T 59: | blue (215: A/A | orange |
| | | | C/T) | 217: C/C 247: | (243: T/G 251: |
| | | | | T/T 251: T/T) | G/G 269: C/C) |
| Jackson LA | grey (251: T/T) | red (251: C/C) | blue (56: T/T 59: | grey (215: A/A | grey (243: T/T |
| | | | C/C) | 217: C/C 247: | 251: A/A 269: |
| | | | | T/T 251: C/T) | T/T) |
| Jenkins | grey (251: T/T) | red (251: C/C) | grey (56: T/T 59: | grey blue (215: | orange |
| | | | T/T) | A/A 217: A/A | (243: T/G 251: |
| | | | | 247: T/T 251: | G/G 269: C/C) |
| | | | | C/T) | |
| Kanza | grey (251: T/T) | grey (251: A/C) | grey (56: T/T 59: | grey (215: A/A | grey (243: T/T |
| | | | T/T) | 217: C/C 247: | 251: A/A 269: |
| | | | | T/T 251: C/T) | T/T) |
| Kiowa | grey (251: T/T) | red (251: C/C) | grey (56: T/T 59: | orange (215: | blue (243: T/T |
| | | | T/T) | C/C 217: C/C | 251: A/A 269: |
| | | | | 247: A/A 251: | C/T) |
| | | | | C/A) | |
| Lakota | red (251: C/C) | red (251: C/C) | red (56: T/T 59: | red (215: A/A | grey (243: T/T |
| | | | C/T) | 217: A/A 247: | 251: A/A 269: |
| | | | | T/T 251: T/T) | T/T) |
| Mahan | blue (251: C/T) | red (251: C/C) | red (56: T/T 59: | aqua (215: C/C | grey (243: T/T |
| | | | C/T) | 217: A/A 247: | 251: A/A 269: |
| | | | | A/A 251: A/T) | T/T) |
| Major | blue (251: C/T) | red (251: C/C) | red (56: T/T 59: | blue (215: A/A | grey (243: T/T |
| | | | C/T) | 217: C/C 247: | 251: A/A 269: |
| | | | | T/T 251: T/T) | T/T) |
| Mohawk | blue (251: C/T) | red (251: C/C) | blue (56: T/T 59: | red (215: A/A | grey (243: T/T |
| | | | C/C) | 217: A/A 247: | 251: A/A 269: |
| | | | | T/T 251: T/T) | T/T) |
| Moreland | blue (251: C/T) | red (251: C/C) | blue (56: T/T 59: | orange (215: | orange |
| | | | C/C) | C/C 217: C/C | (243: T/G 251: |
| | | | | 247: A/A 251: | G/G 269: C/C) |
| | | | | C/A) | |
| Nacono | blue (251: C/T) | red (251: C/C) | grey (56: T/T 59: | blue (215: A/A | red (243: T/G |
| | | | T/T) | 217: C/C 247: | 251: G/G 269: |
| | | | | T/T 251: T/T) | C/C) |
| Oconee | blue (251: C/T) | red (251: C/C) | blue (56: T/T 59: | blue (215: A/A | blue (243: T/T |
| | | | C/C) | 217: C/C 247: | 251: A/A 269: |
| | | | | T/T 251: T/T) | C/T) |
| Odom | blue (251: C/T) | red (251: C/C) | red (56: T/T 59: | grey (215: A/A | blue (243: T/T |
| | | | C/T) | 217: C/C 247: | 251: A/A 269: |
| | | | | T/T 251: C/T) | C/T) |
| Pawnee | red (251: C/C) | red (251: C/C) | red (56: T/T 59: | red (215: A/A | red (243: T/G |
| | | | C/T) | 217: A/A 247: | 251: G/G 269: |
| | | | | T/T 251: T/T) | C/C) |
| Philema | grey (251: T/T) | red (251: C/C) | grey (56: T/T 59: | grey (215: A/A | blue (243: T/T |
| | | | T/T) | 217: C/C 247: | 251: A/A 269: |
| | | | | T/T 251: C/T) | C/T) |
| Podsednik | grey (251: T/T) | red (251: C/C) | red (56: T/T 59: | blue (215: A/A | grey (243: T/T |
| | | | C/T) | 217: C/C 247: | 251: A/A 269: |
| | | | | T/T 251: T/T) | T/T) |
| Riverside | grey (251: T/T) | red (251: C/C) | grey (56: T/T 59: | green (215: A/A | grey (243: T/T |
| | | | T/T) | 217: C/C 247: | 251: A/A 269: |
| | | | | T/T 251: C/C) | T/T) |
| Russell | grey (251: T/T) | red (251: C/C) | grey (56: T/T 59: | grey (215: A/A | green (243: T/T |
| | | | T/T) | 217: C/C 247: | 251: A/G 269: |
| | | | | T/T 251: C/T) | T/T) |
| Schley | blue (251: C/T) | red (251: C/C) | red (56: T/T 59: | red (215: A/A | orange |
| | | | C/T) | 217: A/A 247: | (243: T/G 251: |
| | | | | T/T 251: T/T) | G/G 269: C/C) |
| Shawnee | blue (251: C/T) | red (251: C/C) | red (56: T/T 59: | red (215: A/A | grey (243: T/T |
| | | | C/T) | 217: A/A 247: | 251: A/A 269: |
| | | | | T/T 251: T/T) | T/T) |
| SHG | blue (251: C/T) | red (251: C/C) | grey (56: T/T 59: | blue (215: A/A | red (243: T/G |
| | | | T/T) | 217: C/C 247: | 251: G/G 269: |
| | | | | T/T 251: T/T) | C/C) |
| Shoshoni | grey (251: T/T) | green (251: A/A) | grey (56: T/T 59: | grey (215: A/A | blue (243: T/T |
| | | | T/T) | 217: C/C 247: | 251: A/A 269: |
| | | | | T/T 251: C/T) | C/T) |
| Sioux | grey (251: T/T) | red (251: C/C) | grey (56: T/T 59: | red (215: A/A | orange |
| | | | T/T) | 217: A/A 247: | (243: T/G 251: |
| | | | | T/T 251: T/T) | G/G 269: C/C) |
| Stuart | grey (251: T/T) | red (251: C/C) | red (56: T/T 59: | green (215: A/A | orange |
| | | | C/T) | 217: C/C 247: | (243: T/G 251: |
| | | | | T/T 251: C/C) | G/G 269: C/C) |
| Success | grey (251: T/T) | red (251: C/C) | red (56: T/T 59: | blue (215: A/A | grey (243: T/T |
| | | | C/T) | 217: C/C 247: | 251: A/A 269: |
| | | | | T/T 251: T/T) | T/T) |
| Sumner | blue (251: C/T) | red (251: C/C) | grey (56: T/T 59: | red (215: A/A | orange |
| | | | T/T) | 217: A/A 247: | (243: T/G 251: |
| | | | | T/T 251: T/T) | G/G 269: C/C) |
| Syrup Mill | grey (251: T/T) | green (251: A/A) | grey (56: T/T 59: | green (215: A/A | blue (243: T/T |
| | | | T/T) | 217: C/C 247: | 251: A/A 269: |
| | | | | T/T 251: C/C) | C/T) |
| VC168 | grey (251: T/T) | red (251: C/C) | grey (56: T/T 59: | blue (215: A/A | grey (243: T/T |
| | | | T/T) | 217: C/C 247: | 251: A/A 269: |
| | | | | T/T 251: T/T) | T/T) |
| W. Schley | blue (251: C/T) | red (251: C/C) | red (56: T/T 59: | grey (215: A/A | blue (243: T/T |
| | | | C/T) | 217: C/C 247: | 251: A/A 269: |
| | | | | T/T 251: C/T) | C/T) |
| Wichita | blue (251: C/T) | red (251: C/C) | red (56: T/T 59: | red (215: A/A | grey (243: T/T |
| | | | C/T) | 217: A/A 247: | 251: A/A 269: |
| | | | | T/T 251: T/T) | T/T) |
| Witte | grey (251: T/T) | red (251: C/C) | green (56: T/G | blue (215: A/A | grey (243: T/T |
| | | | 59: C/T) | 217: C/C 247: | 251: A/A 269: |
| | | | | T/T 251: T/T) | T/T) |
| Zinner | grey (251: T/T) | red (251: C/C) | red (56: T/T 59: | orange (215: | orange |
| | | | C/T) | C/C 217: C/C | (243: T/G 251: |
| | | | | 247: A/A 251: | G/G 269: C/C) |
| | | | | C/A) | |

TABLE 6

| | Primer ID | | | | |
|---|---|---|---|---|---|
| | CI0058 | CI0062 | CI0081 | CI0084 | CI0089 |
| Putative target | GO: 0016301 kinase | GO: 0003824 | no assembly or | no assembly or | no assembly or |
| Gene based on | activity, GO: 0005524 | catalytic activity | target gene | target gene | target gene |
| black walnut | ATP binding, | | | | |
| annotation | GO: 0005525 GTP | | | | |
| | binding, GO: 0006614 | | | | |
| | SRP-dependent | | | | |
| | cotranslational | | | | |
| | protein targeting to | | | | |
| | membrane | | | | |
| Pecan ORTET | 3 | 4 | 4 | 4 | 4 |
| grouping | | | | | |
| Alley | grey (251: A/G) | red (235: T/T | red (215: A/A | red (235: A/G | red (251: G/A |
| | | 251: C/C) | 236: C/C 250: | 251: A/T) | 329: C/C) |
| | | | A/C) | | |
| Amling | grey blue (251: G/G) | grey (235: T/T | grey (215: A/A | green (235: A/A | red (251: G/A |
| | | 251: C/T) | 236: C/C 250: | 251: A/T) | 329: C/C) |
| | | | C/C) | | |
| Apache | grey blue (251: G/G) | grey (235: T/T | red (215: A/A | red (235: A/G | green (251: G/G |
| | | 251: C/T) | 236: C/C 250: | 251: A/T) | 329: C/C) |
| | | | A/C) | | |
| Barton | red (251: A/A) | red (235: T/T | grey (215: A/A | blue (235: A/A | red (251: G/A |
| | | 251: C/C) | 236: C/C 250: | 251: T/T) | 329: C/C) |
| | | | C/C) | | |
| Brooks | grey blue (251: G/G) | orange (235: | grey (215: A/A | red (235: A/G | red (251: G/A |
| | | T/T 251: T/T) | 236: C/C 250: | 251: A/T) | 329: C/C) |
| | | | C/C) | | |
| Burkett | grey blue (251: G/G) | red (235: T/T | orange (215: A/C | red (235: A/G | green (251: G/G |
| | | 251: C/C) | 236: A/C 250: | 251: A/T) | 329: C/C) |
| | | | A/A) | | |
| Byrd | red (251: A/A) | green (235: C/T | Negative | grey (235: G/G | red (251: G/A |
| | | 251: C/C) | | 251: A/A) | 329: C/C) |
| Caddo | grey blue (251: G/G) | orange (235: | grey (215: A/A | blue (235: A/A | red (251: G/A |
| | | T/T 251: T/T) | 236: C/C 250: | 251: T/T) | 329: C/C) |
| | | | C/C) | | |
| Candy | grey (251: A/G) | grey (235: T/T | grey (215: A/A | red (235: A/G | grey (251: A/A |
| | | 251: C/T) | 236: C/C 250: | 251: A/T) | 329: C/C) |
| | | | C/C) | | |
| Cape Fear | grey (251: A/G) | red (235: T/T | orange (215: A/C | grey (235: G/G | grey (251: A/A |
| | | 251: C/C) | 236: A/C 250: | 251: A/A) | 329: C/C) |
| | | | A/A) | | |
| Carmichael | grey (251: A/G) | red (235: T/T | aqua (215: A/A | grey (235: G/G | red (251: G/A |
| | | 251: C/C) | 236: C/C 250: | 251: A/A) | 329: C/C) |
| | | | A/C) | | |
| Carter | grey (251: A/G) | red (235: T/T | grey (215: A/A | grey (235: G/G | red (251: G/A |
| | | 251: C/C) | 236: C/C 250: | 251: A/A) | 329: C/C) |
| | | | C/C) | | |
| Cheyenne | red (251: A/A) | red (235: T/T | grey (215: A/A | red (235: A/G | red (251: G/A |
| | | 251: C/C) | 236: C/C 250: | 251: A/T) | 329: C/C) |
| | | | C/C) | | |
| Choctaw | red (251: A/A) | grey (235: T/T | grey (215: A/A | red (235: A/G | red (251: G/A |
| | | 251: C/T) | 236: C/C 250: | 251: A/T) | 329: C/C) |
| | | | C/C) | | |
| Clark | red (251: A/A) | red (235: T/T | grey (215: A/A | grey (235: G/G | grey (251: A/A |
| | | 251: C/C) | 236: C/C 250: | 251: A/A) | 329: C/C) |
| | | | C/C) | | |
| Creek | grey blue (251: G/G) | red (235: T/T | grey (215: A/A | grey (235: G/G | grey (251: A/A |
| | | 251: C/C) | 236: C/C 250: | 251: A/A) | 329: C/C) |
| | | | C/C) | | |
| Curtis | grey blue (251: G/G) | grey (235: T/T | grey (215: A/A | red (235: A/G | grey (251: A/A |
| | | 251: C/T) | 236: C/C 250: | 251: A/T) | 329: C/C) |
| | | | C/C) | | |
| Dependable 6-2 | grey blue (251: G/G) | red (235: T/T | grey (215: A/A | grey (235: G/G | red (251: G/A |
| | | 251: C/C) | 236: C/C 250: | 251: A/A) | 329: C/C) |
| | | | C/C) | | |
| Dependable 8-3 | grey (251: A/G) | red (235: T/T | grey (215: A/A | grey (235: G/G | red (251: G/A |
| | | 251: C/C) | 236: C/C 250: | 251: A/A) | 329: C/C) |
| | | | C/C) | | |
| Desirable 18-18 | grey blue (251: G/G) | grey (235: T/T | grey (215: A/A | red (235: A/G | green (251: G/G |
| | | 251: C/T) | 236: C/C 250: | 251: A/T) | 329: C/C) |
| | | | C/C) | | |
| Desirable 5-2 | grey blue (251: G/G) | grey (235: T/T | grey (215: A/A | red (235: A/G | green (251: |
| | | 251: C/T) | 236: C/C 250: | 251: A/T) | G/G 329: C/C) |
| | | | C/C) | | |
| Eclipse | grey (251: A/G) | red (235: T/T | grey (215: A/A | grey (235: G/G | red (251: G/A |
| | | 251: C/C) | 236: C/C 250: | 251: A/A) | 329: C/C) |
| | | | C/C) | | |
| Elliott | grey blue (251: G/G) | red (235: T/T | blue (251: A/C | green (235: A/A | grey (251: A/A |
| | | 251: C/C) | 272: A/C 286: | 251: A/T) | 329: C/C) |
| | | | A/C) | | |
| Evers | Negative | green (235: C/T | Negative | blue (235: A/A | grey (251: A/A |
| | | 251: C/C) | | 251: T/T) | 329: C/C) |
| Excel | red (251: A/A) | red (235: T/T | grey (215: A/A | blue (235: A/A | red (251: G/A |
| | | 251: C/C) | 236: C/C 250: | 251: T/T) | 329: C/C) |
| | | | C/C) | | |
| Forkert | grey blue (251: G/G) | red (235: T/T | red (215: A/A | grey (235: G/G | red (251: G/A |
| | | 251: C/C) | 236: C/C 250: | 251: A/A) | 329: C/C) |
| | | | A/C) | | |
| Gafford | grey blue (251: G/G) | red (235: T/T | blue (251: A/C | grey (235: G/G | red (251: G/A |
| | | 251: C/C) | 272: A/C 286: | 251: A/A) | 329: C/C) |
| | | | A/C) | | |
| Giles | red (251: A/A) | grey (235: T/T | grey (215: A/A | green (235: A/A | red (251: G/A |
| | | 251: C/T) | 236: C/C 250: | 251: A/T) | 329: C/C) |
| | | | C/C) | | |
| Halbert | grey (251: A/G) | green (235: C/T | blue (251: A/C | grey (235: G/G | red (251: G/A |
| | | 251: C/C) | 272: A/C 286: | 251: A/A) | 329: C/C) |
| | | | A/C) | | |
| Humble | grey blue (251: G/G) | red (235: T/T | grey (215: A/A | grey (235: G/G | red (251: G/A |
| | | 251: C/C) | 236: C/C 250: | 251: A/A) | 329: C/C) |
| | | | C/C) | | |
| Jackson BW | grey blue (251: G/G) | grey (235: T/T | red (215: A/A | red (235: A/G | red (251: G/A |
| | | 251: C/T) | 236: C/C 250: | 251: A/T) | 329: C/C) |
| | | | A/C) | | |
| Jackson LA | red (251: A/A) | red (235: T/T | grey (215: A/A | red (235: A/G | red (251: G/A |
| | | 251: C/C) | 236: C/C 250: | 251: A/T) | 329: C/C) |
| | | | C/C) | | |
| Jenkins | red (251: A/A) | red (235: T/T | grey (215: A/A | grey (235: G/G | grey (251: A/A |
| | | 251: C/C) | 236: C/C 250: | 251: A/A) | 329: C/C) |
| | | | C/C) | | |
| Kanza | grey (251: A/G) | grey (235: T/T | grey (215: A/A | grey (235: G/G | grey (251: A/A |
| | | 251: C/T) | 236: C/C 250: | 251: A/A) | 329: C/C) |
| | | | C/C) | | |
| Kiowa | grey (251: A/G) | grey (235: T/T | grey (215: A/A | red (235: A/G | red (251: G/A |
| | | 251: C/T) | 236: C/C 250: | 251: A/T) | 329: C/C) |
| | | | C/C) | | |
| Lakota | grey (251: A/G) | grey (235: T/T | red (215: A/A | grey (235: G/G | grey (251: A/A |
| | | 251: C/T) | 236: C/C 250: | 251: A/A) | 329: C/C) |
| | | | A/C) | | |
| Mahan | red (251: A/A) | grey (235: T/T | red (215: A/A | red (235: A/G | grey (251: A/A |
| | | 251: C/T) | 236: C/C 250: | 251: A/T) | 329: C/C) |
| | | | A/C) | | |
| Major | grey blue (251: G/G) | red (235: T/T | grey (215: A/A | red (235: A/G | blue (251: A/A |
| | | 251: C/C) | 236: C/C 250: | 251: A/T) | 329: C/T) |
| | | | C/C) | | |
| Mohawk | grey (251: A/G) | grey (235: T/T | red (215: A/A | grey (235: G/G | red (251: G/A |
| | | 251: C/T) | 236: C/C 250: | 251: A/A) | 329: C/C) |
| | | | A/C) | | |
| Moreland | grey (251: A/G) | grey (235: T/T | red (215: A/A | grey (235: G/G | grey (251: A/A |
| | | 251: C/T) | 236: C/C 250: | 251: A/A) | 329: C/C) |
| | | | A/C) | | |
| Nacono | grey (251: A/G) | grey (235: T/T | grey (215: A/A | blue (235: A/A | red (251: G/A |
| | | 251: C/T) | 236: C/C 250: | 251: T/T) | 329: C/C) |
| | | | C/C) | | |
| Oconee | red (251: A/A) | grey (235: T/T | red (215: A/A | blue (235: A/A | red (251: G/A |
| | | 251: C/T) | 236: C/C 250: | 251: T/T) | 329: C/C) |
| | | | A/C) | | |
| Odom | red (251: A/A) | grey (235: T/T | grey (215: A/A | blue (235: A/A | green (251: |
| | | 251: C/T) | 236: C/C 250: | 251: T/T) | G/G 329: C/C) |
| | | | C/C) | | |
| Pawnee | red (251: A/A) | red (235: T/T | red (215: A/A | red (235: A/G | red (251: G/A |
| | | 251: C/C) | 236: C/C 250: | 251: A/T) | 329: C/C) |
| | | | A/C) | | |
| Philema | grey blue (251: G/G) | grey (235: T/T | grey (215: A/A | red (235: A/G | green (251: G/G |
| | | 251: C/T) | 236: C/C 250: | 251: A/T) | 329: C/C) |
| | | | C/C) | | |
| Podsednik | grey blue (251: G/G) | red (235: T/T | grey (215: A/A | grey (235: G/G | red (251: G/A |
| | | 251: C/C) | 236: C/C 250: | 251: A/A) | 329: C/C) |
| | | | C/C) | | |
| Riverside | grey blue (251: G/G) | red (235: T/T | grey (215: A/A | green (235: A/A | grey (251: A/A |
| | | 251: C/C) | 236: C/C 250: | 251: A/T) | 329: C/C) |
| | | | C/C) | | |
| Russell | grey (251: A/G) | green (235: C/T | grey (215: A/A | green (235: A/A | green (251: |
| | | 251: C/C) | 236: C/C 250: | 251: A/T) | G/G 329: C/C) |
| | | | C/C) | | |
| Schley | grey (251: A/G) | grey (235: T/T | red (215: A/A | green (235: A/A | red (251: G/A |
| | | 251: C/T) | 236: C/C 250: | 251: A/T) | 329: C/C) |
| | | | A/C) | | |
| Shawnee | grey (251: A/G) | red (235: T/T | red (215: A/A | blue (235: A/A | green (251: |
| | | 251: C/C) | 236: C/C 250: | 251: T/T) | G/G 329: C/C) |
| | | | A/C) | | |
| SHG | grey (251: A/G) | red (235: T/T | grey (215: A/A | red (235: A/G | grey (251: A/A |
| | | 251: C/C) | 236: C/C 250: | 251: A/T) | 329: C/C) |
| | | | C/C) | | |
| Shoshoni | red (251: A/A) | grey (235: T/T | grey (215: A/A | Negative | red (251: G/A |
| | | 251: C/T) | 236: C/C 250: | | 329: C/C) |
| | | | C/C) | | |
| Sioux | grey blue (251: G/G) | grey (235: T/T | grey (215: A/A | green (235: A/A | red (251: G/A |
| | | 251: C/T) | 236: C/C 250: | 251: A/T) | 329: C/C) |
| | | | C/C) | | |
| Stuart | grey (251: A/G) | red (235: T/T | grey (215: A/A | red (235: A/G | grey (251: A/A |
| | | 251: C/C) | 236: C/C 250: | 251: A/T) | 329: C/C) |
| | | | C/C) | | |
| Success | grey (251: A/G) | red (235: T/T | grey (215: A/A | grey (235: G/G | green (251: G/G |
| | | 251: C/C) | 236: C/C 250: | 251: A/A) | 329: C/C) |
| | | | C/C) | | |
| Sumner | grey (251: A/G) | grey (235: T/T | red (215: A/A | green (235: A/A | grey (251: A/A |
| | | 251: C/T) | 236: C/C 250: | 251: A/T) | 329: C/C) |
| | | | A/C) | | |
| Syrup Mill | grey blue (251: G/G) | grey (235: T/T | grey (215: A/A | grey (235: G/G | grey (251: A/A |
| | | 251: C/T) | 236: C/C 250: | 251: A/A) | 329: C/C) |
| | | | C/C) | | |
| VC168 | grey blue (251: G/G) | red (235: T/T | grey (215: A/A | blue (235: A/A | green (251: G/G |
| | | 251: C/C) | 236: C/C 250: | 251: T/T) | 329: C/C) |
| | | | C/C) | | |
| W. Schley | grey blue (251: G/G) | red (235: T/T | red (215: A/A | red (235: A/G | grey (251: A/A |
| | | 251: C/C) | 236: C/C 250: | 251: A/T) | 329: C/C) |
| | | | A/C) | | |
| Wichita | red (251: A/A) | green (235: C/T | orange (215: A/C | blue (235: A/A | grey (251: A/A |
| | | 251: C/C) | 236: A/C 250: | 251: T/T) | 329: C/C) |
| | | | A/A) | | |
| Witte | grey (251: A/G) | red (235: T/T | grey (215: A/A | red (235: A/G | grey (251: A/A |
| | | 251: C/C) | 236: C/C 250: | 251: A/T) | 329: C/C) |
| | | | C/C) | | |
| Zinner | grey (251: A/G) | grey (235: T/T | grey (215: A/A | blue (235: A/A | red (251: G/A |
| | | 251: C/T) | 236: C/C 250: | 251: T/T) | 329: C/C) |
| | | | C/C) | | |

The specific primers suitable to distinguish each of the pecan cultivars are described below. Although detection by “color” is detailed below, Tables 4, 5 and 6 include the actual insertion/deletion or single nucleotide polymorphism(s) corresponding to each color. It will be understood that detection by “color” or actual sequence of the insertion/deletion or single nucleotide polymorphism(s) can be used to distinguish the pecan cultivars as follows:

1. Russell is green group with primer CI0053.

2. Curtis is orange group with primer CI0048.

3. Mahan is aqua group using CI0051.

4. Jenkins is grey blue using CI0051.

5. Major is blue using CI0089.

6. Elliott, Gafford and Halbert are in the blue group with primer CI0081. Primer CI0048 distinguishes Elliott (green), Gafford (blue) and Halbert (red).

7. Wichita, Burkett and Cape Fear are in the orange group with primer CI0081. Primer CI0084 distinguishes Wichita (blue), Burkett (red) and Cape Fear (grey).

8. Byrd, Evers, Halbert, Russell and Wichita are in the green group with primer CI0062. Primer CI0001 identifies Evers (grey), Byrd (orange) and Halbert (red) from Russell and Wichita (orange).

9. Alley, Candy, Riverside, Stuart and Syrup Mill are in the green group with primer CI0051. Primer CI0025 separates Riverside (red), Alley and Stuart (orange), and Candy and Syrup Mill (grey). Primer CI0001 separates Alley (red) and Stuart (blue), Candy (red) and Syrup Mill (orange).

10. Carmichael is in the aqua group with primer CI0081. Primer CI0001 separates Carmichael (red), Byrd (orange) and Evers (grey).

11. Apache, Jackson BW, Caddo, Cape Fear, Forkert, Jenkins, Moreland, Schley, Sioux, Stuart, Sumner and Zinner with Primer CI0053 are in the orange group. Primer CI0012 distinguishes between Apache and Jackson BW (blue) from the rest of the cultivars (red and green). Primer CI0008B distinguishes between Apache (red) and Jackson BW (grey blue). Primer CI0015 also distinguishes Apache (red) and Jackson BW (grey). Primer CI0018 is another option to distinguish Apache (blue) and Jackson BW (grey).

12. Kanza, Curtis and Elliott are in the grey group using CI0043. Primer CI0018 distinguishes between Curtis (blue), Elliot (red) and Kanza (grey). Also, primer CI0025 distinguishes between Curtis (orange), Elliott (red) and Kanza (grey). Primer CI0051 is another option to distinguish between Curtis (blue), Elliott (red) and Kanza (grey). Primer CI0084 also distinguishes between Curtis (red), Elliott (green) and Kanza (grey).

13. Candy, Kanza, Syrup Mill and Witte are in the grey group using CI0025. Primer CI0001 can distinguish between Candy (red), Kanza (grey), Syrup Mill (orange) and Witte (blue).

14. Elliott and Witte are in the green group using CI0048. Primer CI0084 can distinguish between Witte (red) and Elliott (green), and Evers (blue) and Dependable 6-2 (grey).

15. Starking Hardy Giant (SHG), Caddo and Clark are the only ones in the green group with primer CI0012. Primer CI0029 distinguishes between Caddo (grey), Clark (red) and Starking Hardy Giant (blue).

16. W. Schley, Brooks, Burkett and Podsednik are in the orange group with primer CI0015. Primer CI0012 distinguishes between W. Schley and Brooks (red), and Burkett and Pod sednik (blue). Primer CI0089 separates W. Schley (grey) from Brooks (red).

17. Stuart, Jackson LA and Witte are in the blue group with primer CI0001. Primer CI0008B distinguishes between Jackson LA (green), Stuart (grey blue), and Witte (blue). Primer CI0048 also separates Jackson LA (blue), Stuart (red), and Witte (green).

18. Brooks and Caddo are the only two genotypes in the orange group using CI0062. CI0001 distinguishes between Caddo (grey) and Brooks (orange). Primer CI0084 also distinguishes between Caddo (blue) and Brooks (red). CI0012 is another alternative to distinguish Caddo (green) and Brooks (red). Finally, CI0053 also distinguishes between Caddo (orange) and Brooks (grey).

19. Giles, Shoshoni, Creek, Cape Fear and Syrup Mill are in the green group using CI0043. Primer CI0081 distinguishes between Cape Fear (orange), Giles, Shoshoni, Creek and Syrup Mill (grey). Primer CI0001 distinguishes between Syrup Mill (orange), Shoshoni and Evers (grey), and Giles and Creek (red). Primer CI008B differentiates Evers and Giles (grey blue), Creek (blue) and Shoshoni (orange).

20. Russell, Shoshoni and Syrup Mill are in the orange group using CI0008B. Primer CI0025 distinguishes between Russell (red), Shoshoni (orange) and Syrup Mill (grey). Also, primer CI0058 distinguishes Russell (grey), Shoshoni (red) and Syrup Mill (grey blue). In addition, primer CI0089 separates Russell (green), Shoshoni (red) and Syrup Mill (grey).

21. Amling, Oconee, Alley, Candy, Carter, Kanza and Major are in the grey group with primer CI0012. Primer CI0001 separates Amling and Oconee (orange group), Carter, Kanza and Major (grey), and Alley and Candy (red group). Primer CI0008B distinguishes between Amling and Kanza (grey blue), Carter (green), Major (red) and Oconee (blue).

22. Kiowa, Moreland and Zinner are the only ones in the orange group using primer CI0051. Primer CI0048 differentiates Kiowa (grey), Moreland (blue) and Zinner (red). Also, primer CI0084 differentiates Kiowa (red), Moreland (grey) and Zinner (blue).

23. Clark, Eclipse, Lakota and Pawnee are in the red group using primer CI0029. Primer CI0084 distinguishes between Pawnee (red), and Clark, Eclipse and Lakota (grey). Primer CI0001 distinguishes between Clark (grey), Eclipse (red) and Lakota (orange). Also, primer CI0051 distinguishes between Clark (grey), Eclipse (blue), and Lakota (red).

24. Excel, Podsednik, Curtis, Giles and Apache are in the blue group with primer CI0018. Primer CI0001 distinguishes Excel, Curtis and Podsednik (orange), from Apache (grey) and Giles (red). Primer CI0048 distinguishes between Excel (grey), Curtis (orange) and Podsednik (red).

25. Nacono, Shawnee, VC168, Barton, Caddo, Oconee, Odom, Wichita, Zinner, Evers and Excel are in the blue group of primer CI00084. Primer CI0001 separates Caddo, Evers, Nacono, Odom, Shawnee, VC168 and Zinner in the grey group while Barton, Excel, Oconee, and Wichita are in the orange group. Primer CI0008B separates Odom (green), Shawnee and Wichita (red), Caddo, Evers, Nacono and Zinner (grey blue), and Barton, Excel, Oconee and VC168 (blue). Primer CI0029, separates Nacono (blue) from Caddo, Evers, Zinner (grey). Primer CI001 distinguishes VC168 (grey) from Barton, Excel and Oconee (orange). Primer CI0015 distinguishes between Barton (grey), Excel (grey blue) and Oconee (red).

26. Schley, Sumner, Forkert, Apache, Caddo, Cape Fear, Jackson BW, Jenkins, Moreland, Sioux, Stuart and Zinner are in the orange group using primer CI0053. Primer CI0001 distinguishes between Stuart (blue), Cape Fear, Jenkins and Sioux (orange), and Apache, Caddo, Forkert, Jackson BW, Moreland, Schley, Sumner and Zinner (grey). Primer CI0051 distinguishes between Cape Fear (blue), Jenkins (grey blue) and Sioux (red). Primer CI0025 separates Caddo, Jackson BW and Sumner (orange), and Apache, Forkert, Moreland, Schley and Zinner (red). Primer CI0012 distinguishes Caddo (green), Jackson BW (blue) and Sumner (red). Primer CI0008B separates Forkert (blue) and Zinner (grey blue), from Apache, Moreland and Schley (red). Primer CI0018 distinguishes between Apache (blue), Moreland (red) and Schley (grey).

27. Cheyenne, Humble, Alley, Barton, Carter, Desirable 18-18, Desirable 5-2, Gafford, Kiowa, Oconee, Odom, Philema, Shoshoni, Syrup Mill and W. Schley are in the blue group with primer CI0053. Primer CI0025 separates Cheyenne, Barton, Carter, Desirable 18-18, Desirable 5-2, Kiowa, Oconee, Odom, Philema (red) from Humble, Alley, Gafford, Shoshoni, W. Schley (orange) and Syrup Mill (grey). Primer CI0018 distinguishes Humble (red) from Alley, Gafford, Shoshoni and W. Schley (grey). Primer CI0051 separates Cheyenne, Barton and Oconee (blue) and Carter, Desirable 18-18, Desirable 5-2, Odom and Philema (grey), and Kiowa (orange). Primer CI001 distinguishes between Cheyenne (grey) and Barton and Oconee (blue). Primer CI0012 separates Barton (red) and Oconee (grey).

28. Choctaw, Byrd, Elliott, Forkert, Giles, Lakota, Mohawk, Pawnee, Schley, Shawnee, Sioux, Sumner and Wichita are in the red group with primer CI0051. Primer CI0048 separates Choctaw, Sioux and Sumner (grey) from Byrd, Forkert, Giles, Lakota, Pawnee, Schley, Shawnee and Wichita (red), Elliott (green) and Mohawk (blue). Primer CI0058 distinguishes between Choctaw (red), Sioux (grey blue) and Sumner (grey).

29. Success, Russell, Desirable 5-2, Desirable 18-18, Philema, Apache, Burkett, Odom, Shawnee and VC168 are in the green group with primer CI0089. Primer CI001 separates accessions into two groups: Group 1=Success, Desirable 5-2, Desirable 18-18, Philema, Russell (orange), and Group 2=Apache, Burkett, Odom, Shawnee and VC168 (grey). Within Group 1, Primer CI0084 distinguishes Success (grey), Russell (green), Desirable 5-2, Desirable 18-18 and Philema (red). Primer CI0012 distinguishes Desirable 5-2 (red) from Desirable 18-18 and Philema (blue). Within Group 2, corresponding to entries in the grey group from Primer CI001, Primer CI0084 separates Apache and Burkett (red) and Odom, Shawnee and VC168 (blue). Primer CI008B distinguishes Apache (red) and Burkett (grey blue), and also distinguishes Odom (green), Shawnee (red) and VC168 (blue).

30. Dependable 6-2 (grey blue) and Dependable 8-3 (grey) are distinguished using primer CI0058. Dependable 6-2 originates from an experimental station in Louisiana while Dependable 8-3 originates from Austin, Tex. Both of these ORTET trees were sampled from the Carya germplasm collection located in College Station, Tex., which are maintained as separate ‘Dependable’ entries due to observed phenotypic differences between them.

31. Philema and Desirable 18-18. These are the only two pecan cultivars in which it is not possible to distinguish between them because they have the same genetic profile at all 16 SNP loci evaluated. ‘Desirable’ is reported as one of the first pecan cultivars developed from a controlled cross. The cross was made in the early 1900's by Carl F. Forkert from Ocean Springs, Jackson County, Miss. The parentage is unknown but may be ‘Success’×‘Jewett’. ‘Desirable’ was introduced around 1915 but was not widely disseminated prior to Forkert's death in 1928. This cultivar would probably have been lost if scions had not been sent to the U.S. Pecan Field Station, in Philema, Ga. in 1925.

The SNPs developed in pecan, a valuable species with limited genomic resources, generated a resource of genome-wide SNPs not previously available. The transcriptome sequencing and HRM analysis were used for SNP discovery and validation of the predicted SNPs and these were then used for germplasm characterization and fingerprinting of pecan cultivars with both HRM and Sanger sequencing. The majority of pecan cultivars represented by the ORTET samples for each cultivar evaluated can be distinguished based on individual SNPs or a combination of SNPs. Opportunities for these approaches include the capacity to define the genetic identity of a pecan tree in an orchard based on a DNA fingerprint that enables growers to make educated decisions about how to manage their orchard, particularly as it relates to disease susceptibility and fungicide applications. Also, interested nurseries can certify the genetics of the trees they are selling and growers can make the choice to purchase DNA-certified or non-certified trees.

Thousands of SNPs were identified in pecan, a relevant horticultural species with limited genomic resources. The validated SNPs were used successfully to characterize specific pecan cultivars based on their genetic fingerprint. Additionally, the developed SNPs can also be used to evaluate the identity of grafted wood from pecan cultivars grown, identify the pecan cultivar of existing orchards at the DNA level, assess the genetic diversity of pecan cultivars, and understand the genetic determinants underlying variation for key traits of value to the pecan breeding programs. The identification of SNPs in pecans, in contrast to other types of sequence polymorphism, may result in differences between coding regions of genes that could be functionally important.

While the invention has been described in connection with specific embodiments thereof, it will be understood that the present invention is capable of further modifications by one of skill in the art. It is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible. The present disclosure is therefore intended to encompass any variations, uses, or adaptations of the invention following, in general, the principles of the invention and including such departures from the present disclosure as come within known or customary practice within the art to which the invention pertains and as may be applied to the essential features herein before set forth.

As used herein, the term “cultivar” will be understood to be the same as “variety,” such that the terms “pecan cultivar” and “pecan variety” will have the same meaning. The term “about” is used herein to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When not used in conjunction closed wording in the claims or specifically noted otherwise, the words “a” and “an” denote “one or more.”

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has” or “includes” one or more steps is not limited to possessing only those one or more steps and also covers other unlisted steps. Similarly, any cell that “comprises,” “has” or “includes” one or more traits is not limited to possessing only those one or more traits and covers other unlisted traits.

All of the materials and methods disclosed and claimed herein can be made and used without undue experimentation as instructed by the above disclosure. Although the materials and methods of this invention have been described in terms of preferred embodiments and illustrative examples, it will be apparent to those of skill in the art that variations can be applied to the materials and methods described herein without departing from the concept, spirit and scope of this invention. All such similar substitutes and modifications apparent to those skilled in the art are deemed to be within the spirit, scope and concept of this invention as defined by the appended claims.

### REFERENCES

Barbazuk, B., S. J. Emrich, H. S. Chen, L. Li, P. S. Schnable. 2007. SNP discovery via 454 transcriptome sequencing. Plant J. 51:910-918.
Beedanagari, S. R., S. K. Dove, B. W. Wood, P. J. Conner. 2005. A first linkage map of pecan cultivars based on RAPD and AFLP markers. TAG. 110:1127-1137.
Bundock P C, Eliott F G, Ablett G, Benson A D, Casu R E, Aitken K S, Henry R J. 2009. Plant Biotechnol J. Targeted single nucleotide polymorphism (SNP) discovery in a highly polyploid plant species using 454 sequencing. 7:347-54.
Chaney, W., Y. Han, C. Rohla, M. J. Monteros, and L. J. Grauke. 2015. Developing molecular marker resources for pecan. Acta Hort. (ISHS) 1070:127-132.
Cheung F, Haas B J, Goldberg S M, May G D, Xiao Y, Town C D. 2006. Sequencing Medicago truncatula expressed sequenced tags using 454 Life Sciences technology. 7:272.
Cho M H, Ciulla D, Klanderman B J, Raby B A, Silverman E K. 2008. High-resolution melting curve analysis of genomic and whole-genome amplified DNA. Clin Chem. 54(12):2055-8.
Crane, H. L., C. A. Reed, and M. N. Wood 1937. Nut Breeding. USDA Yearbook of Agriculture: 827-889.
deSilva D, Blackett J. 2007. Assay: High-resolution melting and unlabeled probes. Gen Eng & Biotech News. 27.
Ganal, M. W., T. Altmann, M. S. Roder. 2009. SNP identification in crop plants. Curr Opin Plant Biol. 12: 211-217.
Ganopoulos I., Tsaballa A., Xanthopoulou A., Madesis P. and Tsaftaris A. 2013. Sweet cherry cultivar identification by high-resolution-melting (HRM) analysis using gene-based SNP markers. Plant Molecular Biology Reporter 31 (3), 763-768.
Grauke, L. J., Iqbal, M. J., Reddy, A. S. and Thompson, T. E. 2003. Developing microsatellite DNA markers in Pecan. J. Amer. Soc. Hort. Sci. 128(3):374-380.
Grauke, L. J. and T. E. Thompson. 1996. Pecans and hickories. In: Janick, J. A. and Moore, J. N. (ed.). Fruit Breeding—III Nuts. pp. 185-239. John Wiley and Sons, Inc.
Han, Y., D. M. Khu, M. J. Monteros. 2012. High resolution melting analysis for SNP genotyping and mapping in tetraploid alfalfa (Medicago sativa L.). Molecular Breeding 29:489-501.
Han, Y., Y. Kang, I. Tones-Jerez, F. Cheung, C. D. Town, P. X. Zhao, M. K. Udvardi, M. J. Monteros. 2011. Genome-wide SNP discovery in tetraploid alfalfa using 454 sequencing and high-resolution melting analysis. BMC Genomics 12:350.
Jenkins, J., B. Wilson, J. Grimwood, J. Schumtz, L. J. Grauke. 2015. Towards a reference pecan genome sequence. ISHS Acta Horticulturae 1070. DOI: 10.17660/ActaHortic.2015.1070.11.
Jia, X. D., T. Wang, M. Zhai, Y. R. Li, Z. R. Guo. 2011. Genetic diversity and identification of Chinese-grown pecan using ISSR and SSR markers. Molecules. 16: 10078-10092.
Kearse, M., R. Moir, A. Wilson, S. Stones-Havas, M. Cheung, S. Sturrock, S. Buxton, A. Cooper, S. Markowitz, C. Duran, T. Thierer, B. Ashton, P. Mentjies, & A. Drummond. 2012. Geneious Basic: an integrated and extendable desktop software platform for the organization and analysis of sequence data. Bioinformatics, 28(12): 1647-1649.
Li, X., A. Acharya, A. D. Farmer, J. A. Crow, A. K. Bharti, R. Kramer, Y. Wei, Y. Han, J. Gou, G. D. May, M. J. Monteros, E. C. Brummer. 2012. Prevalence of single nucleotide polymorphism among 27 diverse alfalfa genotypes as assessed by transcriptome sequencing. BMC Genomics 13: 568. doi: 10.1186/1471-2164-13-568.
McHatton, T. H. 1957. The history, distribution and naming of the pecan (Hicoria pecan). Proceedings of Southeastern Pecan Growers Association: 10-34.
Novaes, E, D. R. Drost, W. G. Farmerie, G. J. Pappas, D. Grattapaglia, R. R. Sederoff, M. Kirst. 2008. High-throughput gene and SNP discovery in Eucalyptus grandis, an uncharacterized genome. BMC Genomics. 9:312.
Pereira L. and Martins-Lopes P. 2015. Vitis vinifera L. Single-Nucleotide Polymorphism Detection with High-Resolution Melting Analysis Based on the UDP-Glucose:Flavonoid 3-O-Glucosyltransferase Gene. J. Agric and Food Chem. 63 (41): 9165-9174. DOI: 10.1021/acs.jafc.5b03463.
SantaLucia J Jr, Allawi H T, Seneviratne P A. 1996. Improved nearest-neighbor parameters for predicting DNA duplex stability. Biochemistry 35 (11): 3555-62.
Thompson, T. 1985. Inheritance of heterodichogamy in pecan (Carya illinoensis). J. of Hered. 76:456-458.
Wang. Z., M. Gerstein, M. Snyder. 2009. RNA-Seq: a revolutionary tool for transcriptomics. Nat. Rev. Genet. 10:57-63.
Wood, B. W., J. A. Payne, and L. J. Grauke. 1990. The rise of the U.S. pecan industry. HortScience 25:721-723.
Wood, B. W., W. Smith Morris, R. E. Worley, P. C. Anderson, T. Thompson, 1997. Reproductive and vegetative characteristics of pecan cultivars. HortScience 32:1028-1033.

What is claimed is:

1. A method of identifying a cultivar of a pecan tree, comprising detecting a polymorphism listed in Table 3 from a sample of nucleic acids from said pecan tree, wherein the presence of said polymorphism identifies said cultivar of said pecan tree.

2. The method of claim 1, wherein said cultivar is Alley, Amling, Apache, Barton, Brooks, Burkett, Byrd, Caddo, Candy, Cape Fear, Carmichael, Carter, Cheyenne, Choctaw, Clark, Creek, Curtis, Dependable 6-2, Dependable 8-3, Desirable 5-2, Eclipse, Elliott, Evers, Excel, Forkert, Gafford, Giles, Halbert, Humble, Jackson BW, Jackson LA, Jenkins, Kanza, Kiowa, Lakota, Mahan, Major, Mohawk, Moreland, Nacono, Oconee, Odom, Pawnee, Podsednik, Riverside, Russell, Schley, Shawnee, Starking Hardy Giant (SHG), Shoshoni, Sioux, Stuart, Success, Summer, Syrup Mill, VC168, W. Schley, Wichita, Witte, or Zinner.

3. The method of claim 1, wherein said polymorphism comprises an insertion/deletion or single nucleotide polymorphism detected by amplification of a first nucleic acid segment comprising said first insertion/deletion or single nucleotide polymorphism from said sample of nucleic acids, and analysis of said first nucleic acid segment by high-resolution melting or sequencing to detect said first insertion/deletion or single nucleotide polymorphism.

4. The method of claim 1, wherein said polymorphism is within a locus in the genome of said pecan tree having a nucleotide sequence of any one of SEQ ID NOs:33-49.

5. The method of claim 4, wherein said polymorphism is flanked by a pair of oligonucleotide primers of sufficient length to hybridize specifically to any one of SEQ ID NOs:33-49.

6. The method of claim 5, wherein said pair of oligonucleotide primers is selected from the group consisting of SEQ ID NO:1 and SEQ ID NO:2, SEQ ID NO:3 and SEQ ID NO:4, SEQ ID NO:5 and SEQ ID NO:6, SEQ ID NO:7 and SEQ ID NO:8, SEQ ID NO:9 and SEQ ID NO:10, SEQ ID NO:11 and SEQ ID NO:12, SEQ ID NO:13 and SEQ ID NO:14, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:23 and SEQ ID NO:24, SEQ ID NO:25 and SEQ ID NO:26, SEQ ID NO:27 and SEQ ID NO:28, SEQ ID NO:29 and SEQ ID NO:30, and SEQ ID NO:31 and SEQ ID NO:32.

7. The method of claim 3, further comprising detection of a second polymorphism by amplification of a second nucleic acid segment comprising said second single nucleotide polymorphism from said sample of nucleic acids, and analysis of said second nucleic acid segment by high-resolution melting or sequencing to detect said second single nucleotide polymorphism.

8. The method of claim 7, further comprising detection of a third single nucleotide polymorphism by amplification of a third nucleic acid segment comprising said third single nucleotide polymorphism from said sample of nucleic acids, and analysis of said third nucleic acid segment by high-resolution melting or sequencing to detect said third single nucleotide polymorphism.

9. A kit for determining a cultivar of a pecan tree, said kit comprising a pair of oligonucleotide primers of sufficient length to hybridize specifically to any one of SEQ ID NOs:33-49, wherein said cultivar is Alley, Amling, Apache, Barton, Brooks, Burkett, Byrd, Caddo, Candy, Cape Fear, Carmichael, Carter, Cheyenne, Choctaw, Clark, Creek, Curtis, Dependable 6-2, Dependable 8-3, Desirable 5-2, Eclipse, Elliott, Evers, Excel, Forkert, Gafford, Giles, Halbert, Humble, Jackson BW, Jackson LA, Jenkins, Kanza, Kiowa, Lakota, Mahan, Major, Mohawk, Moreland, Nacono, Oconee, Odom, Pawnee, Podsednik, Riverside, Russell, Schley, Shawnee, Starking Hardy Giant (SHG), Shoshoni, Sioux, Stuart, Success, Summer, Syrup Mill, VC168, W. Schley, Wichita, Witte, or Zinner.

10. The kit of claim 9, wherein said kit comprising the following oligonucleotide primer sets: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; SEQ ID NO:15 and SEQ ID NO:16; SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; and SEQ ID NO:31 and SEQ ID NO:32.

11. The kit of claim 11, further comprising instructions for determining said cultivar of said pecan tree.
